SEQUENCE LISTING (2 sequences)

SEQ ID NO: 1 - DNA, 42 bp, Artificial Sequence
  Probe
atctacgaattcatcagggctaaagagtgcagagttacttag

SEQ ID NO: 2 - DNA, 14 bp, Artificial Sequence
  Probe
gttggtgtggttgg

(19) United States
(12) Patent Application Publication
(10) Pub. No.: US 2013/0109029 A1
(43) Pub. Date: May 2, 2013

(54) Methods And Compositions For Cellular Imaging And Cancer Cell Detection Using Light Harvesting Conjugated Polymer-Biomolecular Conjugates

(71) Applicants: Bin Liu, Singapore (SG); Kanyi Pu, Singapore (SG); Kai Li, Singapore (SG); Liping Cai, Singapore (SG); Yanyan Wang, Singapore (SG); Dan Ding, Singapore (SG)

(72) Inventors: Bin Liu, Singapore (SG); Kanyi Pu, Singapore (SG); Kai Li, Singapore (SG); Liping Cai, Singapore (SG); Yanyan Wang, Singapore (SG); Dan Ding, Singapore (SG)

(73) Assignee: NATIONAL UNIVERSITY OF SINGAPORE, Singapore, SG (SG)

(21) Appl. No.: 13/806,447

(22) PCT Filed: Jun. 29, 2011
(86) PCT No.: PCT/SG11/00229

§ 371 Date: Dec. 21, 2012

Related U.S. Application Data
(60) Provisional application No. 61/359,737, filed on Jun. 29, 2010.
(60) Provisional application No. 61/487,880, filed on May 19, 2011.

(51) Int. Cl. C07K 17/02

(52) U.S. Cl. CPC C07K 17/02
USPC 435/7.1; 530/391.1; 530/367

(57) ABSTRACT

The present invention relates to conjugated polyelectrolyte (CPE) or oligoelectrolyte (COE) compounds represented by general structural formulae (I)-(IV), or a salt thereof and methods of using these compounds to detect targets in samples. In particular, the methods include: (1) exposing a sample to a compound of structural formula (I), (II) or (IV) or a salt thereof, allowing the compound to bind to a target and detecting a signal produced by the compound; (2) functionalizing a solid support with a ligand, incubating the sample with a charged CPE or COE and detecting the fluorescence of the solid support and thereby detecting the target or (3) functionalizing a surface of a solid support with a charged ligand, thereby creating a charge on the surface of the solid support; incubating the ligand-functionalized solid support with a sample, whereupon binding of the target, the charge on the surface of the solid support switches; incubating the sample with CPE or COE that has a complementary charge to the charge of the target-bound surface; and detecting the fluorescence of the solid support and thereby detecting the target. The compounds of the present invention possess high photoluminescence quantum yields in biological media, low cytotoxicity, and excellent environmental stability and photostability and can be used in biosensor and bioimaging applications.

### RELATED APPLICATIONS

This application claims the benefit of U.S. Provisional Application No. 61/359,737, filed on Jun. 29, 2010 and U.S. Provisional Application No. 61/487,880, filed on May 19, 2011. The entire teachings of the above applications are incorporated herein by reference.

### BACKGROUND OF THE INVENTION

Fluorescent cellular probes with high selectivity and sensitivity are of central importance not only for fundamental biology and pathophysiology, but also for clinical diagnosis and therapy. Various materials including organic fluorophores, fluorescent proteins and semiconductor quantum dots (QDs) have been extensively applied for cellular imaging. However, each of these materials has disadvantages (e.g., low photobleaching thresholds for organic and genetic fluorophores, severe cytotoxicity for QDs under oxidative conditions, and, for live cell imaging, microinjection or electroporation techniques are often necessary to deliver the fluorescent probes). Recently, conjugated polyelectrolytes (CPEs) with π-electron delocalized backbones and water-soluble side chains have provided a versatile platform for biological sensing and imaging. In particular, primary investigation using CPEs as simple nonspecific stains reveals that they possess low cytotoxicity, good photostability and sufficient brightness for use as cellular probes.

Conjugation of fluorescent materials to antibodies that have a specific affinity to receptors in target cells is a general method of constructing cellular probes. However, implementation of such a strategy using CPEs appears to be difficult because strong nonspecific electrostatic and hydrophobic interactions, which can significantly influence bioconjugation reactions, exist between CPEs and biomolecules. These interactions could also depress the selectivity of the obtained probes. As a result, there is a need for selective, fluorescent cellular probes based on CPE-antibody conjugates.

### SUMMARY OF THE INVENTION

One embodiment of the invention is a compound represented by any one of Structural Formulas (I)-(IV), or a salt thereof, wherein the values and alternative values for the variables are as defined in the Detailed Description of the Invention.

Another embodiment of the invention is a method of detecting a target in a sample, comprising exposing a sample to a compound of Structural Formula (I), (II) or (IV), or a salt thereof, wherein the values and alternative values for the variables in Structural Formulas (I), (II) and (IV) are as defined in the Detailed Description of the Invention; allowing the compound to bind to a target; and detecting a signal produced by the compound, thereby detecting the target.

Another embodiment of the invention is a method of detecting a target in a sample, comprising functionalizing a solid support with a ligand; incubating the ligand-functionalized solid support with a sample; incubating the sample with a charged conjugated polyelectrolyte (CPE) or charged conjugated oligoelectrolyte (COE); and detecting the fluorescence of the solid support, thereby detecting the target.

Yet another embodiment of the invention is a method of detecting a target in a sample, comprising functionalizing a surface of a solid support with a charged ligand, thereby creating a charge on the surface of the solid support; incubating the ligand-functionalized solid support with a sample, whereupon binding of the target, the charge on the surface of the solid support switches; incubating the sample with a conjugated polyelectrolyte (CPE) or a conjugated oligoelectrolyte (COE) that has a complementary charge to the charge of the target-bound surface; and detecting the fluorescence of the solid support, thereby detecting the target.

The compounds of the invention possess high photoluminescence quantum yields in biological media, low cytotoxicity, and excellent environmental stability and photostability, and can be used in biosensor and bioimaging applications.

### BRIEF DESCRIPTION OF THE FIGURES

The foregoing will be apparent from the following more particular description of example embodiments of the invention, as illustrated in the accompanying figures.

FIG. 1 is a synthetic route to P2.

FIG. 2 is time-resolved confocal laser scanning microscopy (CLSM) fluorescence images of SKBR-3 breast cancer cells stained by affibody-attached P2 under laser scanning for (a) 0 min and (b) 15 minutes.

FIG. 3 is CLSM fluorescence and fluorescence/transmission overlapped images of SKBR-3 (a and b), MCF-7 (c and d), and NIH-3T3 cells (e and f) treated with affibody-attached P2 (0.5 μM) for 20 minutes at 4° C.

FIG. 4 is a synthetic route to FA-functionalized CPE-g-PEG (P4.1).

FIG. 5 is a TEM (a) and Tapping-mode atomic force microscopy (AFM) image with cross-sectional analysis (b) of P4.1-assembled nanoparticles (inset shows the enlarged picture of the nanoparticles).

FIG. 6 is CLSM (a) fluorescence and (b) fluorescence/transmission overlapped images of MCF-7 cells stained by P3.1; CLSM (c) fluorescence and (d) fluorescence/transmission overlapped images of MCF-7 cells, and (e) fluorescence and (f) fluorescence/transmission overlapped images of NIH-3T3 cells stained by P4.1. Excitation at 543 nm (5% laser power) and collection of fluorescence signals above 650 nm.

FIG. 7 shows the chemical structure of P3-phalloidin conjugate.

FIG. 8 is confocal image of Hela cells under continuous excitation (λₘₐₓ=488 nm) after 0 min (A) and 15 min (B) and the fluorescence/transmission overlapped images of the corresponding cells (C, D) taken after incubation with P3-phalloidin for 2 h at 0.5 μM.

FIG. 9 is a 3D sectional CLSM image of Hela cells after incubation with P3-phalloidin conjugate for 2 h at 0.5 μM.

FIG. 10 is CLSM images of Hela cells after incubation with P3-phalloidin conjugate for 2 h at 0.5 μM.

FIG. 11 is a synthetic route to dye-attached HCPE-PEG.

FIG. 12 shows the chemical structure of bimodal HCPE.

FIG. 13 is in vivo non-invasive fluorescence images of H₂₂ tumor-bearing mice after intravenous injection of Gd(III)-labeled HCPE NPs.

FIG. 14 is T1-weighted MR images of H₂₂ tumor-bearing mice after intravenous injection of Gd(III)-labeled HCPE NPs at 0 (left image) and 3 (right image) hours post-injection (the dotted line indicates the tumor site).

FIG. 15 is a schematic illustration of CPE-based, label-free protein detection.

FIG. 16 is an absorbance spectrum of PFVSO₃ in water at [RU]=4 μM (excitation at 428 nm).

FIG. 17 is a graph depicting the photoluminescence intensity (triangle) and percentage of unbound lysozyme (square) as a function of surface density of aptamers on silica nanoparticle (NP) surface.

FIG. 18 is a photoluminescence (PL) spectrum of polymer-stained NPs incubated with (a) 20 μg/mL lysozyme; (b) a mixture of 20 μg/mL each for BSA, thrombin, and trypsin; or (c) a mixture of (a) and (b) followed by subsequent staining with 1 μM PFVSO₃Na in 15 mM PBS at pH=7.4 (excitation at 428 nm).

FIG. 19 is a PL spectra of polymer-stained NPs incubated with increasing concentrations of lysozyme in 15 mM PBS at pH=7.4 (excitation at 428 nm).

FIG. 20 is the calibration curves for lysozyme detection plotted as PL intensity as a function of lysozyme concentration (each data point represents the average value of six independent experiments with error bars indicated).

### DETAILED DESCRIPTION OF THE INVENTION

A description of example embodiments of the invention follows.

As used herein, the singular forms “a,” “an” and “the” include plural referents unless the context clearly dictates otherwise. Thus, for example, reference to “a biomolecule” can include a plurality of biomolecules. Further, the plurality can comprise more than one of the same biomolecule or a plurality of different biomolecules.

As used herein, “conjugated polyelectrolyte,” “conjugated oligoelectrolyte,” “CPE” and “COE” refer to fluorescent macromolecules with electron-delocalized backbones and water-soluble side chains. CPEs and COEs combine the light-harvesting properties of conjugated polymers with the electrostatic behavior of electrolytes, providing unique opportunities for construction of sensory and imaging materials.

As used herein, “oligo” refers to a monomer unit repeating ten or less times in the chain. For example, “oligo(ethylene oxide)” refers to an ethylene oxide repeat unit [e.g., —(CH₂CH₂O)ₙ], wherein n is 1-10; 2-10; 2-5; 5-10; 2-8; 2-6; or 3-6.

As used herein, “poly” refers to a monomer unit repeating ten or more times in the chain. For example, “poly(ethylene oxide)” refers to an ethylene oxide repeat unit [e.g., —(CH₂CH₂O)ₙ], wherein n is greater than 10. Specifically, n is 10-100, 10-200; 10-50; 10-15; or 50-100.

In some embodiments of the invention, the CPEs and COEs are functionalized with polyhedral oligomeric silsesquioxanes (POSS). As used herein, “polyhedral oligomeric silsesquioxanes” or “POSS” are a category of polycyclic compounds, which consist of a silicon/oxygen cage surrounded by tunable organic substitution groups. Due to the nano-scaled dimension and facile modification of substitution groups, POSS serve as organic-inorganic nanobuilding blocks for the construction of fluorescent nanomaterials. Functionalization with POSS can minimize self-quenching of CPEs and COEs, which can be desirable for optical applications.

### Compounds of the Invention

In a first embodiment of the invention, the CPE or COE is a hyperbranched CPE (HCPE). Specifically, the HCPE is represented by Structural Formula (I):

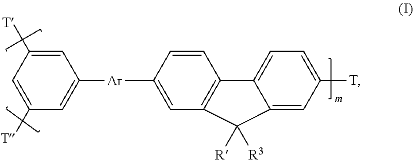

or a salt thereof; wherein:
R′ and R³ are each independently hydrogen or a charged side group;
m is an integer between 2 and 50, inclusive;
Ar is an optionally substituted monocyclic or polycyclic aromatic ring system or an optionally substituted monocyclic or polycyclic heteroaromatic ring system; and
T, T′ and T″ are each independently a terminating group, -L or -L′-B, wherein L and L′ are each independently a linking group and wherein B, for each occurrence, is independently a biomolecule.

As used herein, “hyperbranched conjugated polyelectrolyte” or “HCPE” refers to a CPE which has a densely branched structure and a large number of end groups.

In a first aspect of the first embodiment, Ar is fluorene, benzene, biphenyl, thiophene, benzothiadiazole, 4,7-di(thien-5′-yl)-2,1,3-benzothiadiazole, pyridine, bipyridinium, triphenylamine, anthracene or carbazole. Specifically, Ar is benzothiadiazole or benzene. More specifically, Ar is benzothiadiazole. The values and alternative values for the remaining variables are as described in the first embodiment or the second embodiment, or aspects thereof.

In a second aspect of the first embodiment, T, T′ and T″ are each a terminating group, -L or -L′-B, wherein L and L′ are each independently a linking group and wherein B, for each occurrence, is independently a biomolecule. In one embodiment, T, T′ and T″ are each —CCH. Alternatively, T, T′ and T″ are each -L′-B, wherein B, for each occurrence, is independently a biomolecule. Alternatively, T, T′ and T″ are each -L′-B, wherein each B is a biomolecule. Alternatively, T, T′ and T″ are each -L′-B, wherein B, for each occurrence, is independently one of two different biomolecules (e.g., a protein, such as streptavidin, or a reporter tag, such as a fluorescent dye molecule), and wherein each of the two different biomolecules is present in the compound. The values and alternative values for the remaining variables are as described in the first embodiment, or the first aspect thereof, or the second embodiment, or aspects thereof.

In a third aspect of the first embodiment, R′ and R³ are each a charged side group selected from the group consisting of —(CH₂)ₙN(R²)₃X, —(OCH₂CH₂)ₙN(R²)₃X and —(CH₂CH₂O)_{q}CH₂CH₂N(R²)₃X, wherein R² is (C1-C6)alkyl, n is an integer between 2 and 13, inclusive, q is an integer between 1 and 12, inclusive, and X is an anionic counterion. The values and alternative values for the remaining variables are as described in the first embodiment, or first or second aspects thereof, or second embodiment or aspects thereof.

In a fourth aspect of the first embodiment, the HCPE is represented by the following structural formula:

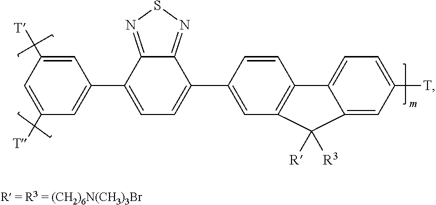

wherein the values and alternative values for the remaining variables are as described in the first embodiment, or first to third aspects thereof, or second embodiment, or aspects thereof.

In a fifth aspect of the first embodiment, m is an integer between 2 and 30, inclusive, wherein the values and alternative values for the remaining variables are as described in the first embodiment, or the first to fourth aspects thereof, or second embodiment, or aspects thereof.

In a sixth aspect of the first embodiment, T, T′ and T″ are each independently -L or -L′-B, wherein each B is a biomolecule and wherein the values and alternative values for the variables are as described in the first embodiment, or first to fifth aspects thereof, or second embodiment, or aspects thereof.

In a seventh aspect of the first embodiment, L is represented by one of the following structural formulas:

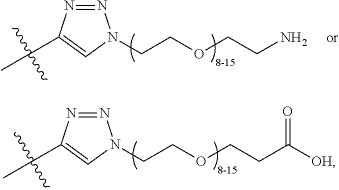

or a salt thereof. The values and alternative values for the variables are as described in the first embodiment, or first to sixth aspects thereof, or second embodiment, or aspects thereof.

In an eighth aspect of the first embodiment, L′ is represented by one of the following structural formulas:

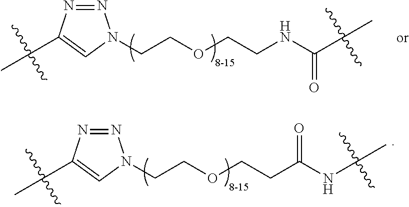

The values and alternative values for the variables are as described in the first embodiment, or first to seventh aspects thereof, or second embodiment, or aspects thereof.

In a ninth aspect of the first embodiment, T, T′ and T″ are each independently a terminating group, wherein the values and alternative values for the variables are as described in the first embodiment, or first to eighth aspects thereof, or second embodiment, or aspects thereof.

A second embodiment of the invention is a molecular brush represented by structural formula (II):

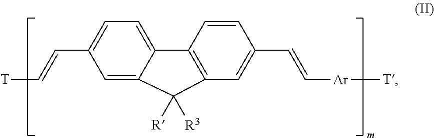

or a salt thereof; wherein:
R′ and R³ are each independently hydrogen or a charged side group, wherein the charged side group is optionally functionalized with -L or -L′-B, wherein L and L′ are each independently a linking group and wherein B, for each occurrence, is independently a biomolecule;
m is an integer between 2 and 50, inclusive;
Ar is an optionally substituted monocyclic or polycyclic aromatic ring system or an optionally substituted monocyclic or polycyclic heteroaromatic ring system; and
T and T′ are each independently a terminating group.

As used herein, “molecular brush” refers to a CPE or COE with densely grafted side chains on a linear polymeric backbone.

In a first aspect of the second embodiment of the invention, R′ and R³ are each a cationic alkyl group or a cationic oligo or poly(ethylene oxide) group functionalized with -L′-B, wherein each B is a biomolecule. The values and alternative values for the variables are as defined in the first embodiments, or aspects thereof, or the second embodiment.

In a second aspect of the second embodiment, T and T′ are each independently hydrogen, halo, —CH═CH₂ or —CH₂CH₃, wherein the values and alternative values for the variables are as defined in the first embodiment, or aspects thereof, or the second embodiment, or first aspect thereof.

In a third aspect of the second embodiment, the CPE or COE is represented by the following structural formula:

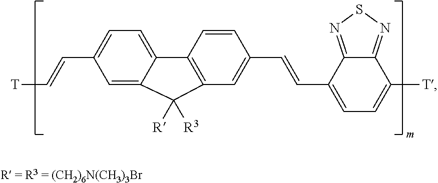

wherein the values and alternative values for the variables are as defined in the first, second or fifth embodiments, or aspects thereof, or the sixth embodiment, or the first or second aspects thereof.

In a fourth aspect of the second embodiment, Ar is an optionally substituted monocyclic or polycyclic (C6-C12) aromatic ring system or an optionally substituted monocyclic or polycyclic (C6-C12)heteroaromatic ring system, wherein the values and alternative values for the remaining variables are as described in the first embodiment, or aspects thereof, or the second embodiment, or the first through third aspects thereof.

In a fifth aspect of the second embodiment, the CPE or COE is not represented by the following structural formula:

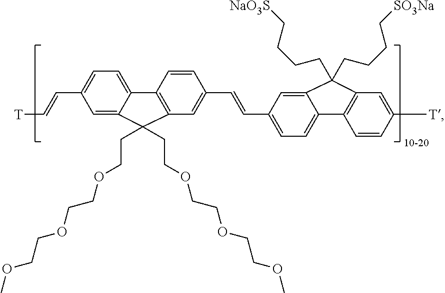

wherein the values and alternative values for the remaining variables are as described in the first embodiment, or aspects thereof, or the second embodiment, or the first through fourth aspects thereof.

In a sixth aspect of the second embodiment, m is an integer between 2 and 10, inclusive, or 20 and 30, inclusive, wherein the values and alternative values for the remaining variables are as described in the first embodiment, or aspects thereof, or the second embodiment, or the first through fifth aspects thereof.

In a seventh aspect of the second embodiment, Ar is fluorene, benzene, biphenyl, thiophene, benzothiadiazole, 4,7-di(thien-5′-yl)-2,1,3-benzothiadiazole, pyridine, bipyridinium, triphenylamine, anthracene or carbazole. Specifically, Ar is benzothiadiazole or anthracene. The values and alternative values for the remaining variables are as described in the first embodiment, or aspects thereof, or the second embodiment, or the first through sixth aspects thereof.

In an eighth aspect of the second embodiment, R′ and R³ are each independently hydrogen or an unfunctionalized charged side group, wherein the values and alternative values for the remaining variables are as described in the first embodiment, or aspects thereof, or the second embodiment, or the first through seventh aspects thereof.

In a ninth aspect of the second embodiment, R′ and R³ are each an unfunctionalized charged side group, wherein the values and alternative values for the remaining variables are as described in the first embodiment, or aspects thereof, or the second embodiment, or the first through eighth aspects thereof.

In a tenth aspect of the second embodiment, Ar is

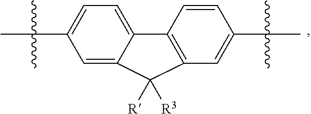

wherein the values and alternative values for the remaining variables are as described in the first embodiment, or aspects thereof, or the second embodiment, or the first through ninth aspects thereof.

In an eleventh aspect of the second embodiment, the charged side groups are selected from the group consisting of —(CH₂)ₙN(R²)₂(R³)X, —(OCH₂CH₂)ₙN(R²)₂(R³)X, and —(CH₂CH₂O)_{q}CH₂CH₂N(R²)₂(R³)X, wherein R² is (C1-C6)alkyl, R³ is (C1-C6)alkyl, (C1-C6)alkenyl, (C1-C6)alkynyl or azido(C1-C6)alkyl, n is an integer between 2 and 13, inclusive, q is an integer between 1 and 12, inclusive, and X is an anionic counterion. Specifically, R³ is (C1-C6)alkynyl or azido(C1-C6)alkyl. More specifically, R³ is (C1-C6)alkynyl. Yet more specifically, R³ is —(CH₂)₂CCH. The values and alternative values for the remaining variables are as described in the first embodiment, or aspects thereof, or the second embodiment, or the first through tenth aspects thereof.

In a twelfth aspect of the second embodiment, L is represented by one of the following structural formulas:

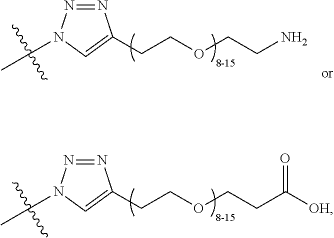

and L′ is represented by one of the following structural formulas:

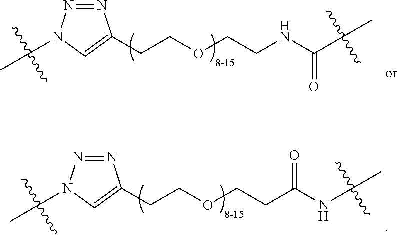

The values and alternative values for the remaining variables are as described in the first embodiment, or aspects thereof, or the second embodiment, or the first through eleventh aspects thereof.

A third embodiment of the invention is a CPE or COE represented by Structural Formula (III):

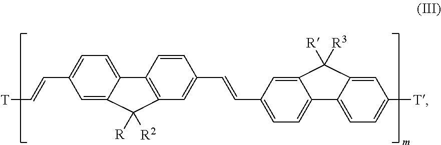

or a salt thereof, wherein:
R and R² are each independently —(OCH₂CH₂)ₚOCH₃ or —(CH₂CH₂O)ₚCH₃, wherein p is an integer between 1 and 100, inclusive;
R′ and R³ are each independently hydrogen or a charged side group;
m is an integer between 2 and 50, inclusive; and
T and T′ are each independently a terminating group.

In a first aspect of the third embodiment, the CPE or COE is represented by Structural Formula (III), or a salt thereof, with the proviso that the CPE or COE is not represented by the following structural formula:

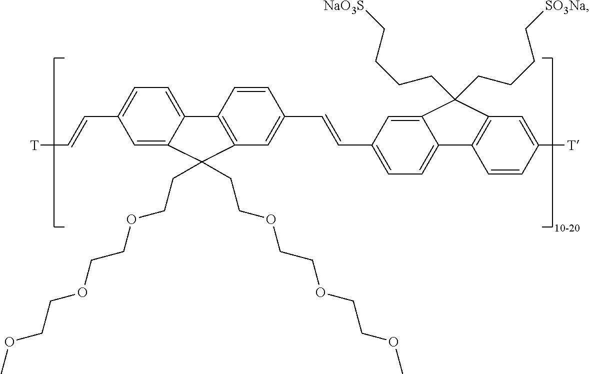

wherein the values and alternative values for the remaining variables are as described in the first or second embodiments, or aspects thereof, or the third embodiment.

In a second aspect of the third embodiment, R and R² are each —(OCH₂CH₂)ₚOCH₃ or —(CH₂CH₂O)ₚCH₃. Specifically, R and R² are each —(CH₂CH₂O)ₚCH₃. More specifically, p is an integer between 1 and 50, inclusive, between, 1 and 25, inclusive, between 1 and 10, inclusive, or between 1 and 5, inclusive or p is 3. The values and alternative values for the remaining variables are as described in the first or second embodiments, or aspects thereof, or the third embodiment, or first aspect thereof.

In a third aspect of the third embodiment, R′ and R³ are each independently a charged side group, wherein the values and alternative values for the remaining variables are as described in the first or second embodiments, or aspects thereof, or the third embodiment, or first or second aspects thereof.

In a fourth aspect of the third embodiment, R′ and R³ are each a charged side group. Specifically, R′ and R³ are each an anionic side group. Alternatively, R′ and R³ are each a cationic side group. The values and alternative values for the remaining variables are as described in the first or second embodiments, or aspects thereof, or the third embodiment, or first through third aspects thereof.

In a fifth aspect of the third embodiment, m is an integer between 2 and 10, inclusive, or 20 and 30, inclusive. Specifically, m is an integer between 2 and 10, inclusive. Alternatively, m is an integer between 20 and 30, inclusive. The values and alternative values for the remaining variables are as described in the first or second embodiments, or aspects thereof, or the third embodiment, or first through fourth aspects thereof.

In a sixth aspect of the third embodiment, p is 3 and R′ and R³ are each —(CH₂)ₙSO₃Y, wherein n is 4 and Y is sodium, wherein the values and alternative values for the variables are as described in the first or second embodiments, or aspects thereof, or the third embodiment, or first to fifth aspects thereof.

In a seventh aspect of the third embodiment, the charged side group is an anionic or cationic alkyl side group, an anionic or cationic oligo(ethylene oxide) side group or an anionic or cationic poly(ethylene oxide) side group, wherein the values and alternative values for the variables are as described in the first or second embodiments, or aspects thereof, or the third embodiment, or first to sixth aspects thereof.

In an eighth aspect of the third embodiment, the charged side group is selected from the group consisting of —(CH₂)ₙN(R²)₃X, —(OCH₂CH₂)ₙN(R²)₃X, —(CH₂CH₂O)_{q}CH₂CH₂N(R²)₃X, —(CH₂)ₙX′, —(OCH₂CH₂)ₙX′, —(OCH₂CH₂)ₙOX′, —(CH₂CH₂O)ₙX′ and —(CH₂CH₂O)_{q}CH₂CH₂X′, wherein R² is (C1-C6)alkyl, n is an integer between 2 and 13, inclusive, q is an integer between 1 and 12, inclusive, X is an anionic counterion and X′ is —CO₂Y, —SO₃Y or —PO₃Y₂, wherein Y is hydrogen or a cationic counterion. The values and alternative values for the variables are as described in the first or second embodiments, or aspects thereof, or the third embodiment, or first to seventh aspects thereof.

In a ninth aspect of the third embodiment, R and R² are each —(OCH₂CH₂)ₚOCH₃ or —(CH₂CH₂O)ₚCH₃; and R′ and R³ are each hydrogen or a charged side group, wherein the values and alternative values for the variables are as described in the first or second embodiments, or aspects thereof, or the third embodiment, or first to eighth aspects thereof.

In a tenth aspect of the third embodiment, the compound is represented by Structural Formula (IIIa):

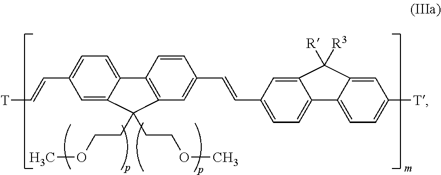

or a salt thereof, wherein the values and alternative values for the variables are as described in the first or second embodiments, or aspects thereof, or the third embodiment, or first to ninth aspects thereof.

In an eleventh aspect of the third embodiment, the compound is represented by the Structural Formula (IIIb):

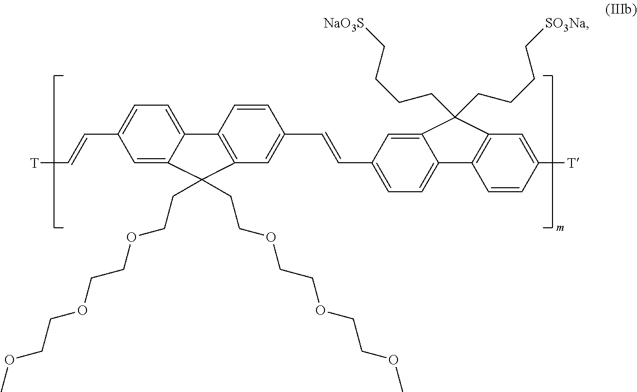

wherein the values and alternative values for the variables are as described in the first or second embodiments, or aspects thereof, or the third embodiment, or first to tenth aspects thereof.

In a fourth embodiment of the invention, the CPE or COE is functionalized with POSS and is represented by the following structural formula:

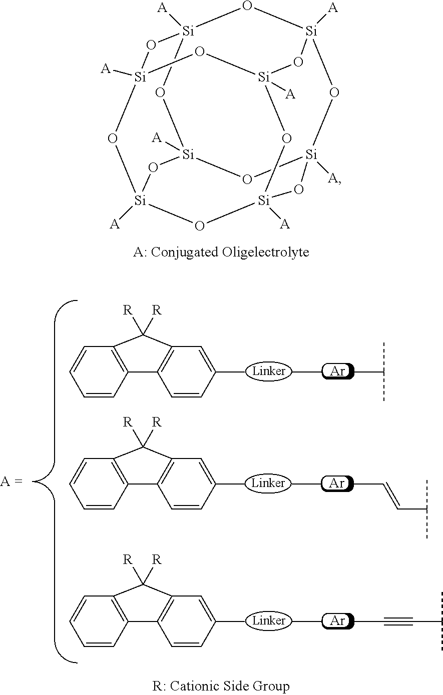

or a salt thereof, wherein:
Ar is an optionally substituted aromatic group;
Linker is a single bond, double bond, triple bond or —CR¹₂—; wherein each R¹ is independently hydrogen, halogen, hydroxy, amino, (C₁-C₆)alkyl, (C₁-C₆)alkenyl, (C₁-C₆)alkynyl, or (C₁-C₆)alkoxy; wherein the alkyl, alkenyl, alkynyl or alkoxy may be optionally substituted with halogen, hydroxy, (C₁-C₄)alkoxy or amino;

each R is independently hydrogen, a cationic alkyl side group or a cationic oligo or poly(ethylene oxide) group.

In a first aspect of the third embodiment, Linker is a single bond, double bond, triple bond, —CH₂— or —CH₂CH₂—, wherein the values and alternative values for the variables are as described in the fourth embodiment or in the fifth embodiment, or aspects thereof.

In a fifth embodiment, the CPE or COE is functionalized with POSS and is represented by the following structural formula:

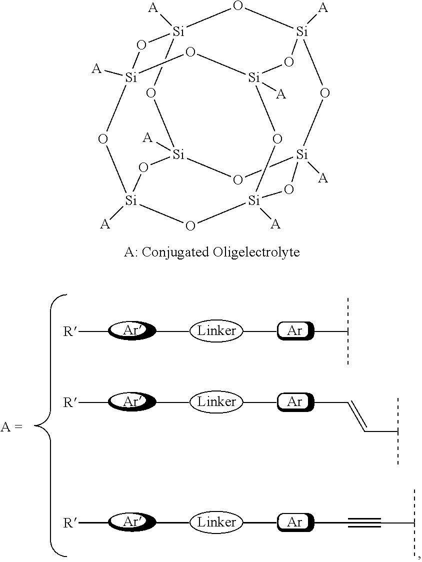

or a salt thereof; wherein:

each

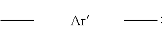

is independently selected from:

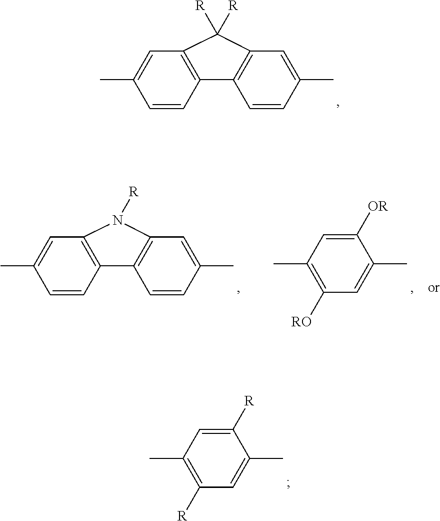

each Ar is independently an optionally substituted aromatic group;

each R is independently a cationic, anionic, or neutral alkyl group or a cationic, anionic, or neutral oligo or poly(ethylene oxide) group;

each Linker is a single bond, double bond, triple bond, —CH₂— or —CH₂CH₂—; and

each R′ is independently a terminating group.

In a first aspect of the fifth embodiment, Ar is fluorene, benzene, biphenyl, pyridine, bipyridinium, triphenylamine, anthracene, thiophene, carbazole, or benzothiadiazole. Optional substituents include those defined by R. The values and alternative values for the remaining variables are as described in the fifth embodiment or in the fourth embodiment, or aspects thereof.

In a second aspect of the fifth embodiment, each R is independently selected from the group consisting of hydrogen, —(CH₂)ₙNMe₃X; —(CH₂)ₙNEt₃X; —(CH₂CH₂O)_{q}CH₂CH₂NMe₃X and —(CH₂CH₂O)_{q}CH₂CH₂NEt₃X, wherein X is an anionic counterion, n is an integer between 2 and 13, inclusive, and q is an integer between 1 and 12, inclusive. Specifically, each R is independently selected from the group consisting of hydrogen, —(CH₂)ₙNMe₃X and —(CH₂CH₂O)_{q}CH₂CH₂NMe₃X, wherein X is an anionic counterion, n is an integer between 2 and 13, inclusive, and q is an integer between 1 and 12, inclusive. The values and alternative values for the remaining variables are as described in the fourth embodiment, or aspects thereof, or in the fifth embodiment, or first aspect thereof.

In a third aspect of the fifth embodiment, the POSS-functionalized CPE or COE is represented by the following structural formula:

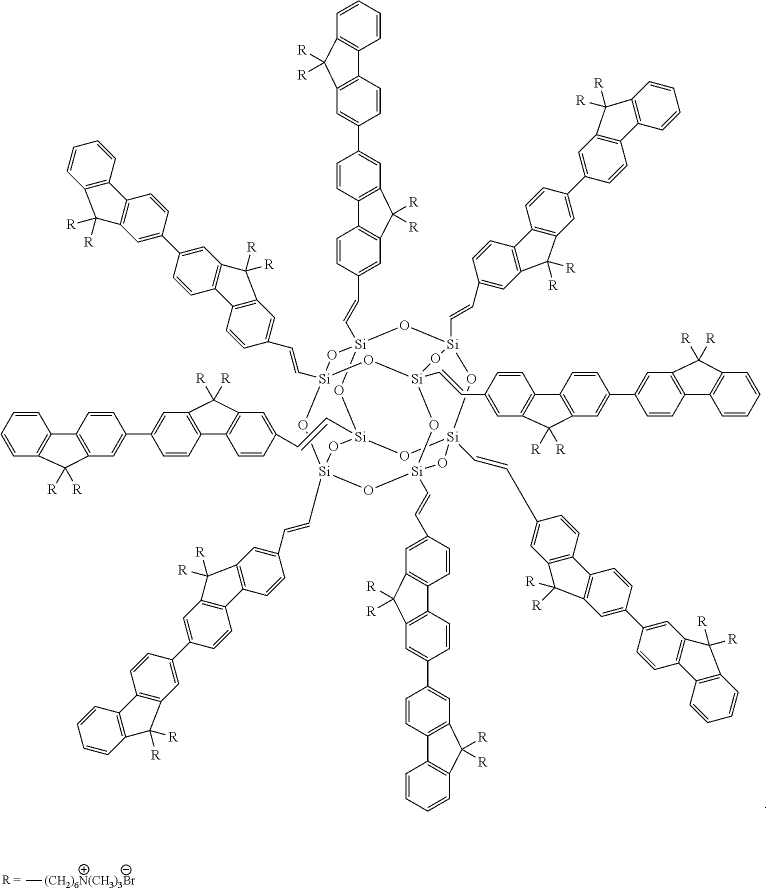

In a fourth aspect of the fifth embodiment, the POSS-functionalized CPE or COE is represented by the following structural formula:

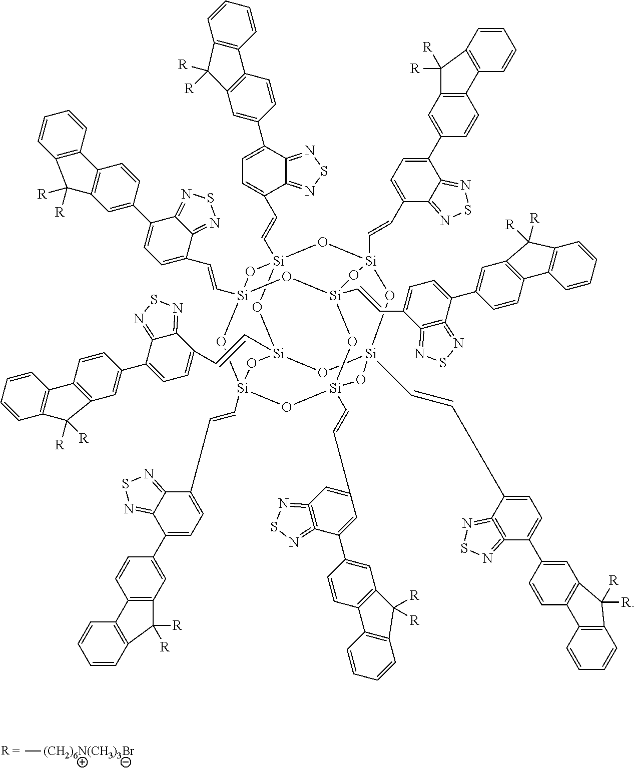

In a fifth aspect of the fifth embodiment, R is an anionic group selected from —(CH₂)ₙX′, —(OCH₂CH₂)ₙX′, —(OCH₂CH₂)ₙOX′, —(CH₂CH₂O)ₙX′ and —(CH₂CH₂O)_{q}CH₂CH₂X′, wherein X′ is selected from —SO₃Y, —PO₃Y₂, and —CO₂Y, n is an integer between 2 and 13, inclusive, q is an integer between 1 and 12, inclusive, and Y is a cationic counterion. The values and alternative values for the remaining variables are as described in the fourth embodiment, or aspects thereof, or the fifth embodiment, or the first through fourth aspects thereof.

In a sixth aspect of the fifth embodiment, each R is selected from —(CH₂)ₙX′, —(OCH₂CH₂)ₙX′, —(OCH₂CH₂)ₙOX′, —(CH₂CH₂O)ₙX′ and —(CH₂CH₂O)_{q}CH₂CH₂X′, wherein X′ is selected from —SO₃Y, —PO₃Y₂, and —CO₂Y, n is an integer between 2 and 13, inclusive, q is an integer between 1 and 12, inclusive, and Y is sodium or potassium. The values and alternative values for the remaining variables are as described in the fourth embodiment, or aspects thereof, or the fifth embodiment, or the first through fifth aspects thereof.

In a sixth embodiment of the invention, the CPE or COE is a compound of Structural Formula (IV):

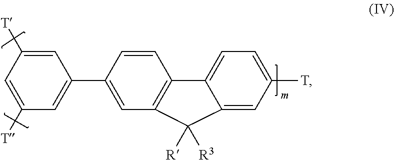

or a salt thereof; wherein:

R′ and R³ are each independently hydrogen or a charged side group;

m is an integer between 2 and 50, inclusive; and

T, T′ and T″ are each independently a terminating group, -L or -L′-B, wherein L and L′ are each independently a linking group and wherein B, for each occurrence, is a biomolecule.

In a first aspect of the sixth embodiment, R′ and R³ are each a charged side group, wherein the values and alternative values for the remaining variables are as described in the first and second embodiments, and aspects thereof, or the sixth embodiment.

In a second aspect of the sixth embodiment, the charged side group is a cationic alkyl group, a cationic oligo or poly(ethylene oxide) group, an anionic alkyl group or an anionic oligo or poly(ethylene oxide) group, wherein the values and alternative values for the remaining variables are as described in the first and second embodiments, and aspects thereof, or the sixth embodiment, or first aspect thereof.

In a third aspect of the sixth embodiment, the charged side group is —(CH₂)₂COOH, wherein the values and alternative values for the remaining variables are as described in the first and second embodiments, and aspects thereof, or the sixth embodiment, or first or second aspects thereof.

In some embodiments of the invention, the charged side group can be a cationic alkyl side group, a cationic oligo(ethylene oxide) side group or a cationic poly(ethylene oxide) side group. As used herein, “a cationic alkyl side group” is a (C1-C15)alkyl that includes a moiety, such as an amine, that confers a positive charge. As used herein, “cationic oligo(ethylene oxide) side group” and “cationic poly(ethylene oxide) side group” refer to a polymer of ethylene oxide that includes a moiety, such as an amine, that confers a positive charge. The amine can be a primary, a secondary, a tertiary or a quaternary amine. Specifically, the amine is a quaternary amine. Alternatively, the amine is a protonated amine.

In some embodiments of the invention, the charged side group can be an anionic alkyl side group, an anionic oligo(ethylene oxide) side group or an anionic poly(ethylene oxide) side group. As used herein, “anionic alkyl side group” refers to a (C1-C15)alkyl that includes a moiety, such as a phosphonate, a sulfonate or a carboxylate, that confers a negative charge. As used herein, “anionic oligo(ethylene oxide) side group” and “anionic poly(ethylene oxide) side group” refer to a polymer of ethylene oxide that includes a moiety, such as a phosphonate, a sulfonate or a carboxylate, that confers a negative charge.

In some embodiments of the invention, the charged side groups are selected from the group consisting of —(CH₂)ₙN(R²)₃X, —(OCH₂CH₂)ₙN(R²)₃X, —(CH₂CH₂O)_{q}CH₂CH₂N(R²)₃X, —(CH₂)ₙX′, —(OCH₂CH₂)ₙX, —(OCH₂CH₂)ₙOX′, —(CH₂CH₂O)ₙX′ and —(CH₂CH₂O)_{q}CH₂CH₂X′, wherein R² is (C1-C6)alkyl, n is an integer between 2 and 13, inclusive, q is an integer between 1 and 12, inclusive, X is an anionic counterion and X is —CO₂Y, —SO₃Y or —PO₃Y₂, wherein Y is hydrogen or a cationic counterion.

In other embodiments of the invention, the charged side groups are selected from the group consisting of —(CH₂)ₙN(R²)₃X, —(CH₂CH₂O)_{q}CH₂CH₂N(R²)₃X, —(CH₂)ₙX′, —(CH₂CH₂O)ₙX′ and —(CH₂CH₂O)_{q}CH₂CH₂X′, wherein R² is (C1-C6)alkyl, n is an integer between 2 and 13, inclusive, q is an integer between 1 and 12, inclusive, X is an anionic counterion and X′ is —CO₂Y, —SO₃Y or —PO₃Y₂, wherein Y is hydrogen or a cationic counterion.

In some embodiments of the invention, the charged side groups are selected from the group consisting of —(CH₂)ₙN(R²)₃X, —(OCH₂CH₂)ₙN(R²)₃X and —(CH₂CH₂O)_{q}CH₂CH₂N(R²)₃X, wherein R² is (C1-C6)alkyl, n is an integer between 2 and 13, inclusive, q is an integer between 1 and 12, inclusive, and X is an anionic counterion. Specifically, R² is methyl or ethyl.

In some embodiments of the invention, the charged side groups are selected from the group consisting of —(CH₂)ₙX, —(OCH₂CH₂)ₙX, —(OCH₂CH₂)ₙOX′, —(CH₂CH₂O)ₙX′ and —(CH₂CH₂O)_{q}CH₂CH₂X′, wherein n is an integer between 2 and 13, inclusive, q is an integer between 1 and 12, inclusive, and X′ is —CO₂Y, —SO₃Y or —PO₃Y₂, wherein Y is hydrogen or a cationic counterion. Specifically, X′ is —SO₃Y or —PO₃Y₂. More specifically, X′ is —SO₃Y. Alternatively, Y is a cationic counterion.

In some embodiments of the invention, the charged side groups are optionally functionalized with -L or -L′-B, wherein L and L′ are each independently a linking group and wherein B, for each occurrence is independently a biomolecule. In a specific embodiment, the charged side groups are functionalized with -L or -L′-B, wherein L and L′ are each a linking group and wherein each B is a biomolecule. In another specific embodiment, the charged side groups are selected from the group consisting of —(CH₂)ₙN(R²)₂(R³)X, —(OCH₂CH₂)ₐN(R²)₂(R³)X, and —(CH₂CH₂O)_{q}CH₂CH₂N(R²)₂(R³)X, wherein R² is (C1-C6)alkyl, R³ is (C1-C6)alkyl, (C1-C6)alkenyl, (C1-C6)alkynyl or azido(C1-C6)alkyl, n is an integer between 2 and 13, inclusive, q is an integer between 1 and 12, inclusive, and X is an anionic counterion. Specifically, the (C1-C6)alkenyl includes a terminal alkene and/or the (C1-C6)alkynyl includes a terminal alkyne. More specifically, the (C1-C6)alkynyl is —(CH₂)₂CCH.

As used herein, “terminating group” refers to the functional group left at each end of a polymer upon termination of the polymerization reaction. Non-limiting examples of terminating groups include hydrogen, halo, —CH═CH₂, —CCH and —CH₂CH₃.

“Linking group,” as used herein, refers to a bifunctional linker that is capable of reacting with a complementary functional group of a compound of Structural Formula (I), (II) or (IV) and capable of reacting with a complementary functional group of a biomolecule, thereby forming a covalent link between the compound of Structural Formula (I), (H) or (IV) and the biomolecule. Typically, a bifunctional linker comprises two functional groups linked via an alkylene or a divalent oligo or poly(ethylene oxide) radical. For examples of commonly used bifunctional linking groups, see Hermanson, Greg T. Bioconjugate Techniques, Second Edition, Academic Press, Inc. (2008).

In some embodiments, the bifunctional linker is a heterobifunctional linker, meaning the linker undergoes one type of chemical reaction with the compound of Structural Formula (I), (II) or (IV) and a different chemical reaction with the biomolecule. For example, a heterobifunctional linker comprising an azide group linked [via an alkyl group or an oligo or poly(ethylene oxide) group] to an amine group can undergo a [3+2] cycloaddition reaction with, for example, an alkyne of a compound of Structural Formula (I), (II) or (IV), and a coupling reaction with, for example, an activated carboxylic acid of a biomolecule.

“L,” used herein, denotes said bifunctional linker after formation of the covalent bond with the compound of Structural Formula (I) or (II) and prior to formation of a covalent bond with the biomolecule. “L′,” used herein, denotes said bifunctional linker after formation of a covalent bond with a biomolecule. For example, if “L” is:

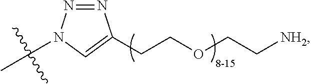

and the complementary functional group of the biomolecule is —COOH, then “L′” is:

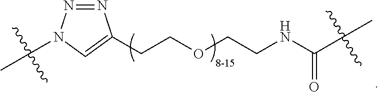

Other functional groups suitable for forming a covalent bond with a compound of Structural Formula (I), (II) or (IV) include functional groups capable of reacting with an alkene, alkyne or halide in a chemical reaction. For example, a diene or an azide could react in a cylcoaddition reaction with an alkene or alkyne. Metal-catalyzed cross-coupling chemistries (e.g., palladium-catalyzed reactions, metathesis reactions) can also be used to form a covalent bond between a functional group of a linker and a compound of Structural Formula (I), (II) or (N).

Functional groups suitable for forming a covalent bond with a biomolecule include, but are not limited to, amino, carboxylate, hydroxyl, thio, haloalkyl, N-hydroxy succinimidyl ester, sulfonato-N-hydroxy succinimidyl ester, thiocyanato, isothiocyanato, nitrophenolyl, iodoacetamidyl, maleimidyl, carboxyl, thioacetyl, sulfonato and phosphoramidityl. Preferably, the functional group is amino, carboxylate, hydroxyl, thio, nitrophenolyl, N-hydroxy succinimidyl ester or sulfonato-N-hydroxy succinimidyl ester. Yet more preferably, the functional group is amino or carboxylate. For examples of functional groups commonly used for bioconjugation, see Hermanson, Greg T. Bioconjugate Techniques, Second Edition, Academic Press, Inc. (2008).

“Alkyl” means a saturated aliphatic branched or straight-chain monovalent hydrocarbon radical having the specified number of carbon atoms. Thus, “(C₁-C₆) alkyl” means a radical having from 1-6 carbon atoms in a linear or branched arrangement. “(C₁-C₆)alkyl” includes, for example, methyl, ethyl, propyl, iso-propyl, n-butyl, tert-butyl, pentyl and hexyl. Typically, alkyl groups have from 1 to 50, 1 to 25, 1 to 15, from 1 to 8, or from 1 to 6 carbon atoms.

“Alkylene” means a saturated aliphatic branched or straight-chain divalent hydrocarbon radical having the specified number of carbon atoms. Thus, “(C₁-C₆)alkylene” means a divalent saturated aliphatic radical having from 1-6 carbon atoms in a linear arrangement, e.g., —[(CH₂)ₙ]—, where n is an integer from 1 to 6, “(C₁-C₆)alkylene” includes methylene, ethylene, propylene, butylene, pentylene and hexylene. Alternatively, “(C₁-C₆)alkylene” means a divalent saturated radical having from 1-6 carbon atoms in a branched arrangement, for example: —[(CH₂CH₂CH₂CH₂CH(CH₃)]—, —[(CH₂CH₂CH₂CH₂C(CH₃)₂]—, —[(CH₂C(CH₃)₂CH(CH₃))]—, and the like. Typically, alkylene has 1 to 50, 1 to 25, 1 to 10 or 1-8 carbon atoms.

“Alkenyl” refers to a straight or branched aliphatic group with at least one double bond. Typically, alkenyl groups have from 2 to 12 carbon atoms, from 2 to 8, from 2 to 6, or from 2 to 4 carbon atoms. Examples of alkenyl groups include ethenyl (—CH═CH₂), n-2-propenyl (allyl, —CH₂CH═CH₂), pentenyl, hexenyl, and the like.

“Alkynyl” refers to a straight or branched aliphatic group having at least 1 site of alkynyl unsaturation. Typically, alkynyl groups contain 2 to 12, 2 to 8, 2 to 6 or 2 to 4 carbon atoms. Examples of alkynyl groups include ethynyl(—C≡CH), propargyl (—CH₂C≡CH), pentynyl, hexynyl, and the like.

As used herein, “halogen” refers to fluorine, chlorine, bromine or iodine. “Halogen” and “halo” are used interchangeably herein.

“Alkoxy” means an alkyl radical attached through an oxygen linking atom. “(C₁-C₃)alkoxy” includes methoxy, ethoxy and propoxy.

“Aryl” or “aromatic” means an aromatic monocyclic or polycyclic (e.g., bicyclic or tricyclic) carbocyclic ring system. Thus, “(C₅-C₁₄)aryl” is a (5-14)-membered monocylic or bicyclic system. Aryl systems include, but are not limited to, phenyl, naphthalenyl, fluorenyl, indenyl, azulenyl, and anthracenyl.

“Hetero” refers to the replacement of at least one carbon atom in a ring system with at least one heteroatom selected from N, S and O. “Hetero” also refers to the replacement of at least one carbon atom in an acyclic system. A hetero ring system or a hetero acyclic system may have, for example, 1, 2 or 3 carbon atoms replaced by a heteroatom.

“Heteroaryl” or “heteroaromatic” means a monovalent heteroaromatic monocyclic or polycyclic (e.g., bicylic or tricyclic) ring radical. A heteroaryl contains 1, 2, 3 or 4 heteroatoms independently selected from N, O and S. Thus, “(C₅-C₁₄)heteroaryl” refers to a (5-14)-membered ring system, wherein at least one carbon atom has been replaced with at least one heteroatom selected from N, S and O. Heteroaryls include, but are not limited to furan, oxazole, thiophene, 1,2,3-triazole, 1,2,4-triazine, 1,2,4-triazole, 1,2,5-thiadiazole 1,1-dioxide, 1,2,5-thiadiazole 1-oxide, 1,2,5-thiadiazole, 1,3,4-oxadiazole, 1,3,4-thiadiazole, 1,3,5-triazine, imidazole, isothiazole, isoxazole, pyrazole, pyridazine, pyridine, pyridine-N-oxide, pyrazine, pyrimidine, pyrrole, tetrazole, and thiazole.

“Bicycloheteroaryl,” as used herein, refers to bicyclic heteroaryl rings, such ase bicyclo[4.4.0] and bicyclo[4.3.0] fused ring systems containing at least one aromatic ring and 1 to 4 heteroatoms independently selected from N, O and S. In some embodiments of the invention, the first ring is a monocyclic heterocyclyl (such as dioxolane) and the second ring is a monocyclic aryl (such as phenyl) or a monocyclic heteroaryl (such as pyridine). Examples of bicyclic heteroaryl rings include, but are not limited to, indole, quinoline, quinazoline, benzothiophene, benzofuran, 2,3-dihydrobenzofuran, benzodioxole, benzimidazole, indazole, benzisoxazole, benzoxazole and benzothiazole.

“Cycloalkyl” means a saturated aliphatic cyclic hydrocarbon ring. Thus, “C₃-C₇ cycloalkyl” means a hydrocarbon radical of a (3-7 membered) saturated aliphatic cyclic hydrocarbon ring. A C₃-C₇ cycloalkyl includes, but is not limited to cyclopropyl, cyclobutyl, cyclopentyl, cyclohexyl and cycloheptyl.

“Heterocyclyl” means a saturated cyclic 4-12 membered aliphatic ring containing 1, 2, 3, 4 or 5 heteroatoms independently selected from N, O or S. When one heteroatom is S, it can be optionally mono- or di-oxygenated (i.e. —S(O)— or —S(O)₂—). The heterocyclyl can be monocyclic, fused bicyclic, bridged bicyclic, spiro bicyclic or polycyclic.

Each aryl and heteroaryl is optionally and independently substituted. Exemplary substituents include halogen, (C₁-C₃)alkoxy, (C₁-C₃)alkylthio, hydroxy, (C₅-C₁₄)aryl, (C₅-C₁₄)heteroaryl, (C₃-C₁₅)cycloalkyl, (C₃-C₁₅)heterocyclyl, amino, (C₁-C₅)alkylamino, (C₁-C₅)dialkylamino, thio, oxo, (C₁-C₅)alkyl, (C₅-C₁₄)aryl(C₁-C₅)alkyl, (C₅-C₁₄)heteroaryl(C₁-C₅)alkyl, nitro, cyano, sulfonato, phosphonato, carboxylate, hydroxyl(C₁-C₅)alkyl and halo(C₁-C₅)alkyl.

Each aryl and heteroaryl can also be optionally and independently substituted with a charged side group which is optionally functionalized with -L or -L′-B, wherein L and L′ are each independently a linking group and wherein B, for each occurrence, is independently a biomolecule.

“Anionic counterion,” as used herein, refers to a negatively charged ion. Examples of anionic counterions include, but are not limited to, halide, trifluoroacetate, acetate, benzenesulfonate, benzoate, perchlorate, sulfonate, bicarbonate, carbonate, citrate, mesylate, methylsulfate, nitrate, phosphate/diphosphate, sulfate, trifluoromethanesulfonate, tetrafluoroborate, ammonium hexafluorophosphate and tetrakis[3,5,-bis(trifluoromethyl)phenyl]borate. Specifically, the anionic counterion is halide, tetrafluoroborate, trifluoromethanesulfonate, ammonium hexafluorophosphate or tetrakis[3,5,-bis(trifluoromethyl)phenyl]borate. More specifically, the halide is bromide or iodide. Yet more specifically, the halide is bromide.

“Cationic counterion,” as used herein, refers to a positively charged ion. Specifically, the cationic counterion is sodium, lithium or potassium. More specifically, the cationic counterion is sodium or potassium. Alternatively, the cationic counterion is a positively charged metal complex, such as cisplatin.

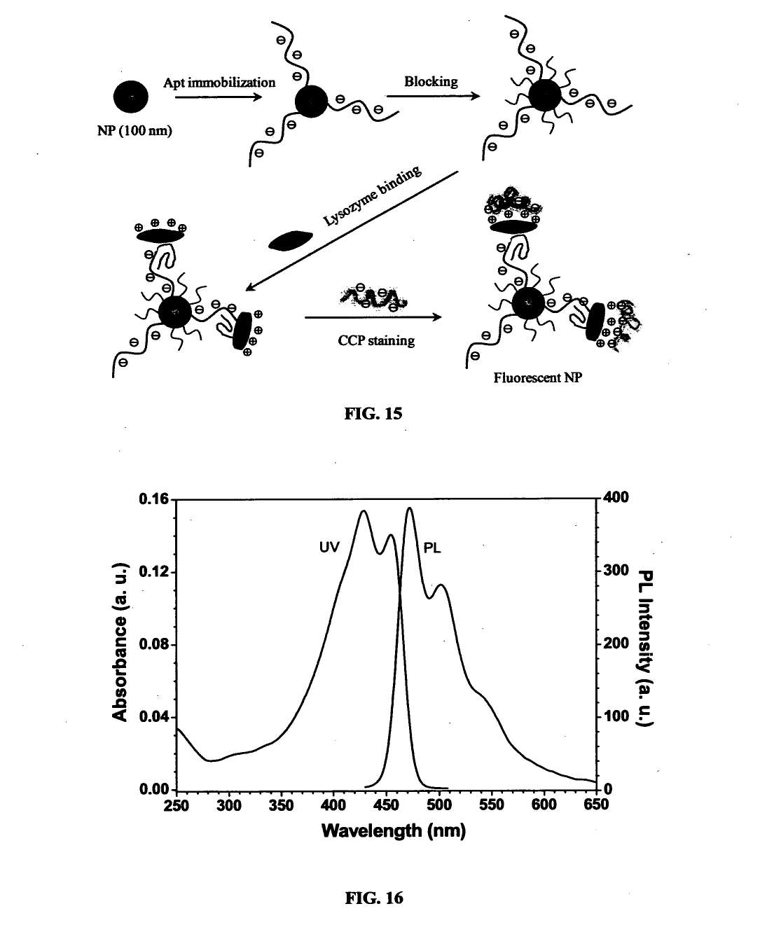

One embodiment of the invention is illustrated in FIG. 15. FIG. 15 depicts the functionalization of NPs [e.g., silica NPs, polystyrene NPs, poly(methylmethacrylate) NPs], with a ligand, such as an aptamer, to yield ligand-functionalized NPs. These ligand-functionalized NPs can be further treated with a blocking agent, such as ethanolamine, to generate blocked NPs. Upon incubation with a sample containing a target, such as a protein (e.g., lysozyme), the blocked NPs specifically bind the target. Binding of the target switches the charge of the NPs. For example, if the NPs were initially negatively-charged, upon binding of the target, the NPs will be positively-charged. A fluorescent CPE that has a complementary charge to the target can be added to the NP-treated sample to yield CPE/target/ligand complexes on the surface of the NP, giving rise to fluorescent NPs after removal of excess CPE, which can be accomplished, for example, by a wash-centrifugation-redispersion process. Since no binding takes place between the ligand and non-specific proteins, the surface charge on the ligand-functionalized NPs that are not bound to the target remains the same as that of the CPE. The CPE is thus electrostatically repelled from NPs not bound to the target and, as a result, NPs not bound to the target remain non-fluorescent. By taking advantage of the recognition-induced switching of surface charge, label-free, naked-eye protein detection can be realized.

“Biomolecule,” as used herein, refers to a natural or synthetic molecule for use in biological systems. Examples of biomolecules include, but are not limited to, proteins, peptides, enzyme substrates, bioactive small molecules, ligands, hormones, antibodies, affibodies, antigens, haptens, carbohydrates, oligosaccharides, polysaccharides, nucleic acids, aptamer, fragments of DNA, fragments of RNA, reporter groups (e.g., fluorescent dyes, contrast reagents) and mixtures thereof.

“Ligand,” as used herein, refers to a molecule that specifically binds to a biomolecule, such as a target. Examples of ligands include, but are not limited to, aptamers [e.g., anti-lysozyme aptamer (5′—NH₂-ATC TAC GAA TTC ATC AGG GCT AAA GAG TGC AGA GTT ACT TAG; SEQ. ID. NO. 1), anti-thrombin aptamer (5′—NH₂-GGT TGG TGT GGT TGG; SEQ. ID. NO. 2)], antibodies (e.g., anti-thrombin), affibodies (e.g., anti-HER2 affibody), proteins (e.g., streptavidin, avidin), and bioactive small molecules (e.g., cisplatin, phalloidin, folic acid).

“Reporter group,” as used herein, refers to a molecule that can be detected in biological systems using spectroscopic techniques [e.g., fluorescence spectroscopy, magnetic resonance imaging (MRI)]. In some embodiments, the reporter group is a fluorescent dye (e.g., AlexaFluor® 555). In other embodiments, the reporter group is a contrast reagent [e.g., diethylenetriaminepentaacetic acid-chelated Gd(III)].

As used herein, “bioactive small molecule” refers to any small molecule that participates in a specific binding interaction with a target. This term is exemplified by metabolites, secondary metabolites, natural products, pharmaceuticals or peptides.

Aptamers are oligonucleic acid or peptide molecules that bind to a specific target molecule. More specifically, aptamers can be classified as: DNA or RNA aptamers, consisting of (usually short) strands of oligonucleotides or peptide aptamers, consisting of a short variable peptide domain, attached at both ends to a protein scaffold. An aptamer to be immobilized on the solid support is selected based upon its ability to bind the biological molecule of interest.

“Target,” as used herein, refers to a biomolecule that specifically binds to another biomolecule. Examples of targets include, but are not limited to, a protein, a peptide, an enzyme, an oligosaccharide, a polysaccharide, a fragment of DNA and a fragment of RNA. In some embodiments of the invention, target proteins (e.g., lysozyme, thrombin) bind ligands (e.g., anti-lysozyme aptamer, anti-thrombin aptamer).

As used herein, “functionalized” refers both to (1) covalent attachment of, for example, a ligand to a nanoparticle, as might be achieved by chemical reaction, and to (2) noncovalent attachment of, for example, a ligand to a nanoparticle, as might be achieved by surface adsorption. In some embodiments, a surface of a solid support (e.g., NP) is functionalized with a ligand. In other embodiments, a linking group is covalently functionalized with a ligand (e.g., phalloidin, folic acid), a reporter group (e.g., a fluorescent dye) or a pharmaceutical (e.g., cisplatin).

The compounds according to the present invention may be in free form or in the form of physiologically acceptable, non-toxic salts. These salts may be obtained by reacting the respective compounds with physiologically acceptable acids and bases. Examples of such salts include but are not limited to hydrochloride, hydrobromide, hydroiodide, hydrofluoride. nitrate, sulfate, bisulfate, pyrosulfate, sulfite, bisulfite, phosphate, acid phosphate, monohydrogenphosphate, dihydrogenphosphate, metaphosphate, pyrophosphate, isonicotinate, acetate, trifluoroacetate, propionate, caprylate, isobutyrate, lactate, salicylate, citrate, tartrate, oxalate, malonate, suberate, sebacate, mandelate, chlorobenzoate, methylbenzoate, dinitrobenzoate, phthalate, phenylacetate, malate, pantothenate, bitartrate, ascorbate, succinate, maleate, gentisinate, fumarate, gluconate, glucuronate, saccharate, formate, benzoate, glutamate, methanesulfonate, ethanesulfonate, benzenesulfonate, p-toluenesulfonate and pamoate (i.e., 1,1′-methylene-bis-(2-hydroxy-3-naphthoate)) salts. Certain compounds of the invention can form pharmaceutically acceptable salts with various amino acids. Suitable base salts include, but are not limited to, aluminium, calcium, lithium, magnesium, potassium, sodium, zinc, and diethanolamine, N,N′-dibenzylethylenediamine, chloroprocaine, choline, dicyclohexylamine, ethylenediamine, N-methylglucamine, and procaine salts.

### Methods Using the Compounds of the Invention

Another embodiment of the invention is a method of detecting a target in a sample, comprising exposing a sample to a compound of Structural Formula (I) or (II), or a salt thereof, wherein the values and alternative values for the variables in Structural Formulas (I) and (II) are as defined in the Detailed Description of the Invention; allowing the compound to bind to a target; and detecting a signal produced by the compound, thereby detecting the target.

In some embodiments, the signal is fluorescence or magnetic resonance.

Another embodiment of this invention is a method of determining the location of a compound in a cell, comprising the steps of exposing the cell to HCPE represented by structural formula (I); allowing the compound to bind to a target within or on the cell; and assaying the cell to determine the location of the compound within or on the cell.

As used herein, “assaying” or “detecting” refers to a determination of the quantity or location, or both of the compounds of this invention in a sample. “Visualizing” is a method of assaying. The compounds of the invention can be used to detect a target in a sample both in vitro (e.g., in a live cell culture or single cell, in fixed cells) or in vivo (e.g., in live subjects, such as mice, humans, rats, and other mammals).

As used herein, a “cell” is any cell with a nucleus. Specifically, the cell is a eukaryotic cell. Further, the cell is a cancer cell. In some embodiments, the cell is a cancer cell and the target is a protein indicative of a cancer, for example, HER2.

The nucleus within a eukaryotic cell can be imaged by the use of the compounds of this invention in bioimaging of live, fixed cells or cell lysates derived thereof from fixed or dead cells. The method comprises the steps of exposing the cell to the compounds of this invention, allowing the compounds of this invention to accumulate within or on the cell, and visualizing the fluorescence emitted from the compounds of this invention. The fluorescence emitted can be assayed by techniques known to those of skill in the art and include, fluorescence, confocal microscopy, two photon fluorescence microscopy, and flow cytometry.

“Fluorescence spectroscopy”, also known as “fluorometry” or “spectrofluorometry,” is a type of electromagnetic spectroscopy which analyzes fluorescence from a sample. A beam of light, usually ultraviolet light, is used to excite the electrons in molecules of certain compounds, causing them to emit light of a lower energy, typically, but not necessarily, visible light.

Typically, fluorescence spectroscopy involves measurement of the different frequencies of fluorescent light that are emitted by a sample, while holding the excitation light at a constant wavelength.

“Two-photon fluorescence spectroscopy” is a type of fluorescence spectroscopy that relies on the quasi-simultaneous absorption of two or more photons (of either the same or different energy) by a molecule.

“Flow cytometry” is a method of counting and sorting cells. A beam of light, usually laser light, is used to excite the electrons in molecules of certain compounds, causing them to emit light of a lower energy.

Typically, fluorescence spectroscopy involves measurement of the different frequencies of fluorescent light that are emitted by a sample, while holding the excitation light at a constant wavelength.

As used herein, “exposing the cell to the compound” means the cell and the compound are present in the same container or in the same solution and may come into contact. Exposing the cell the compound includes adding the compound, either in solution or as a solid, to the culture media used to cultivate the cells.

CPEs with Aptamer-Functionalized Silica Nanoparticles

CPEs undergo a photophysical property change upon interaction with proteins. For example, the emission intensity, emission maximum, and/or the absorption maximum, as well as the associated fluorescence and absorbance profiles, can change upon interaction with proteins. (See (a) Ambade, A. V., et al., S. Polym. Int. 2007, 56, 474-481. (b) Ho, H. A., et al., Acc. Chem. Res. 2008, 41, 168-178. (c) Li, K.; Liu, B. Polym. Chem. 2010, 1, 252-259.)

Water solubility of CPEs is achieved through introduction of charged hydrophilic functionalities to the macromolecular backbone. Good water solubility minimizes polymer interchain aggregation, which leads to less fluorescence quenching and greater fluorescence intensity in aqueous solution. (See (a) Khan, A., et al., Chem. Commun. 2005, 584-586. (b) Lee, K. W., et al., Chem. Commun. 2006, 1983-1985; the entire teachings of which are incorporated herein by reference). In addition, good polymer water solubility can minimize nonspecific interactions between CPEs and the nanoparticles, thereby decreasing any background signal.

One embodiment of the present invention is a method of detecting a target in a sample, comprising: functionalizing a solid support with a ligand; incubating the ligand-functionalized solid support with a sample; incubating the sample with a CPE or COE; and detecting the fluorescence of the solid support, thereby detecting the target. Specifically, the CPE or COE is a charged CPE or COE. In some embodiments, the CPE or COE is a compound represented by Structural Formula (II), (IIIa) or (IIIb). In other embodiments, the CPE or COE is a compound described in the fourth or fifth embodiments of the invention, or aspects thereof. In yet other embodiments, the CPE or COE is a compound described in the first or second embodiment of the invention, or aspects thereof.

A sample can be, for example, a cellular lysate, a biomolecule, a cell, a mixture of biomolecules, or a mixture thereof. A sample can be in the form of a solution in buffer, for example, and can include biological media.

As used herein, “incubating the sample with a CPE or COE” means the sample and the CPE or COE are present in the same container or in the same solution and may come into contact. Incubating the sample with the CPE or COE includes adding the CPE or COE, either in suspension or as a solid, to the sample.

In some embodiments, the method further includes isolating the solid support from the sample. In other embodiments, the method further includes isolating the solid support from the sample and washing the solid support. Isolating the solid support from the sample and/or washing the solid support can occur before detecting the fluorescence of the solid support.

Suitable solid supports include nanoparticles (NPs) or solid-state substrates (e.g., paper, glass, quartz). Silica NPs, in particular, can be easily functionalized, are chemically inert, and are easily separable from biological media. The chemical modification of silica NPs can be accomplished chemically using reactive functional groups (e.g., cyanuric chloride, aldehyde, and NHS ester) (see, for example, Steinberg, G., et al., Biopolymers 2004, 73, 597-605; Kato, N.; Caruso, F. J. Phys. Chem. B 2005, 109, 19604-19612; and Liang, Y, et al., Talanta 2007, 72, 443-449, the entire teachings of each are incorporated herein by reference). Meanwhile, the high density of silica (1.96 g/cm³) facilitates easy separation of NPs from biological media via centrifugation-washing-redispersing circles. Such a method can help to eliminate nonspecific proteins, while retaining the bound target, and can promote the trace detection of a target in biological samples. In addition, silica NPs of 100 nm in diameter are transparent in dilute solutions, and their optical properties do not interfere with those of fluorescent dyes or CPEs.

Aptamer-functionalized silica NPs can be an effective platform for selectively capturing a target, such as lysozyme or thrombin, and effectively isolating the target via centrifugation-washing-redispersing circles. Lysozyme binding to aptamer-functionalized silica NPs switches the surface charges of Apt-NP from negative to partially positive, which subsequently allows for CPE binding, which can be detected as blue-green fluorescence by, for example, the naked eye or a fluorescence spectrometer. Moreover, the linear intensity increase of polymer emission as a function of lysozyme concentration allows the accurate quantification of lysozyme in the concentration range of 0 to approximately 22.5 μM with a limit of detection of approximately 0.36 μg/mL. The high quantum yield and good water solubility of CPEs also enables naked-eye lysozyme detection with picomole sensitivity.

In a specific embodiment, the ligand is an aptamer of SEQ. ID. NO.: 1 and the target is lysozyme. For example, aptamer-functionalized silica nanoparticles (NPs) can capture lysozyme, resulting in a switching of the surface charge from negative to partially positive. The aptamer/protein binding event can be monitored by fluorescence spectroscopy. Upon its addition, PFVSO₃ binds to and “stains” the protein/aptamer/NP complexes via an electrostatic interaction. The blue-green fluorescence of PFVSO₃ can be observed in the presence of lysozyme by the naked eye, while no fluorescence is obtained for NPs treated with a non-specific mixture of proteins.

One embodiment of the invention is a method of detecting a target in a sample, comprising functionalizing a surface of a solid support with a charged ligand, thereby creating a charge (e.g., a positive or negative charge) on the surface of the solid support; incubating the ligand-functionalized solid support with a sample, whereupon binding of the target, the charge on the surface of the solid support switches (e.g., from positive to negative or from negative to positive); incubating the sample with a conjugated polyelectrolyte (CPE) or a conjugated oligoelectrolyte (COE) that has a complementary charge to the charge of the target-bound surface (i.e., if the target-bound surface is negatively charged, the CPE or COE is positively charged and visa versa); and detecting the fluorescence of the sample, thereby detecting the target.

In some embodiments, the ligand is a charged ligand. As used herein, “charged ligand” refers to a ligand having a net positive or net negative charge under the conditions of the assay. Typically, the conditions are neutral conditions or neutral pH. Proteins, CPEs and COEs can also be described as “charged” if they have a net positive or net negative charge under the conditions of the assay.

In a specific embodiment, the biological molecule to be detected is lysozyme, which has an isoelectric point (pI) of 11.0, and is, therefore, positively charged at neutral pH. Lysozyme is a ubiquitous protein serving as the “body's own antibiotic” by cleaving acetyl groups in the polysaccharide walls of many bacteria. Therefore, the lysozyme level in blood is regarded as the clinical index for many diseases such as HIV, myeloid leukemia, etc. (see (a) Vocadlo, D. J., et al., Nature 2001, 412, 835-838. (b) Lee-Huang, S. et al., Proc. Natl. Acad. Sci. U.S.A. 1999, 96, 2678-2681, the teachings of each are herein incorporated by reference).

One embodiment of the invention is a label-free, naked-eye lysozyme detection method using aptamer-functionalized silica NPs as the recognition element to capture a target and an anionic conjugated polymer as “a polymeric stain” to transduce a signal.

### EXEMPLIFICATION

### Example 1

### Synthesis of Biomolecule-Functionalized HCPEs

An affibody-attached hyperbranched conjugated polyelectrolyte (HCPE) was used for targeted fluorescence imaging of human epidermal growth factor receptor 2 (HER2) positive cancer cells. Early-stage detection of HER2 is of clinical significance in personalizing cancer treatment, because HER2 expression levels are closely related to tumor behavior and clinical outcome. Anti-HER2 affibody instead of commonly-used HER2-specific antibody (herceptin) was chosen as the recognition element, in view of its higher affinity for HER2 and smaller size (approximately 7 kDa) compared to herceptin (approximately 150 KDa). The HCPE (P2) used for bioconjugation was endowed with a unique core-shell molecular architecture to minimize nonspecific interactions with biomolecules and to facilitate bioconjugation and targeted cellular imaging.

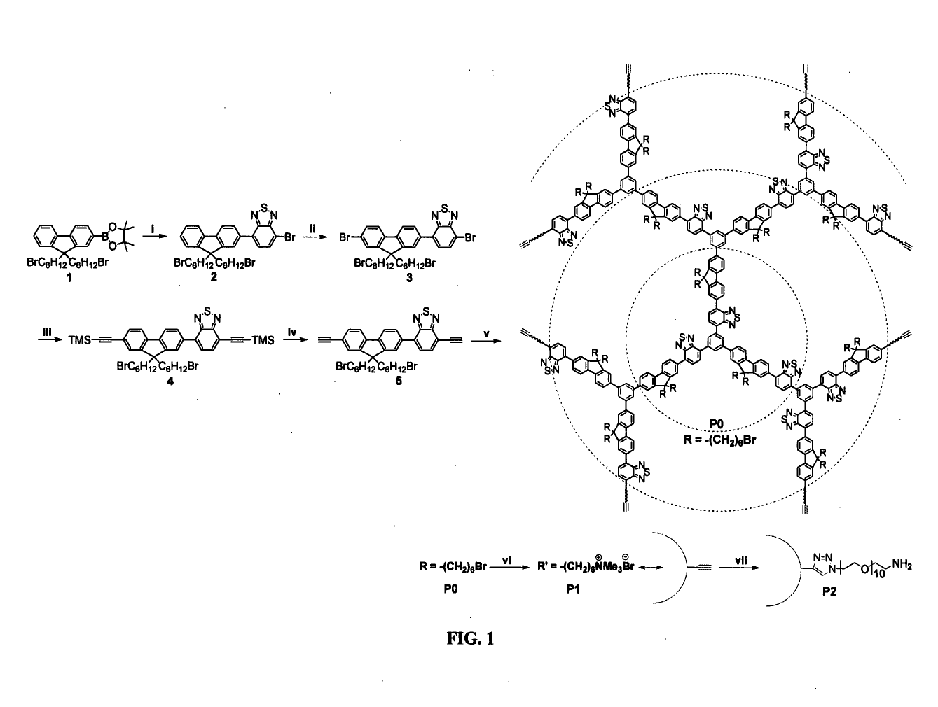

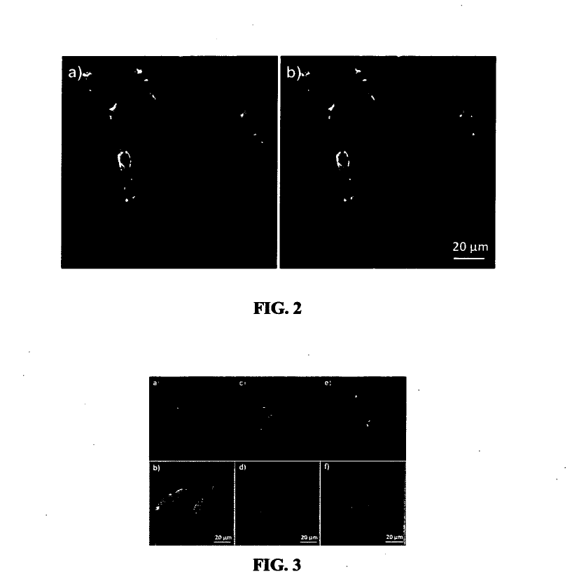

The core-shell HCPE (P2) had a hyperbranched conjugated polymer as the fluorescent core and linear poly(ethylene glycol) (PEG) chains as the protective shell and was synthesized by combining alkyne polycyclotrimerization and alkyne-azide click chemistry. To gain long-wavelength emission that reduced the autofluorescent interference, 4-(9,9′-bis(6-bromohexyl)-7-ethynylfluorenyl)-7-ethynylbenzothiadiazole (5) was the monomer for polycyclotrimerization. The detailed synthesis of P2 is depicted in FIG. 1. Homo-polycyclotrimerization of 5 led to the neutral hyperbranched polymer (P0), which had terminal alkyne groups for later use in a click reaction. Subsequent quaternization of P0 yields its cationic counterpart (P1). Click chemistry was conducted to attach NH₂—PEG to the periphery of P1, affording the core-shell HCPE (P2).

The double-layered HCPE (P2) self-assembled into core-shell nanospheres in aqueous solution, which results in not only enhanced PL quantum yield as compared to P1 but also minimized nonspecific biological interactions to facilitate bioconjugation and targeted cellular imaging. In light of the variability of core-shell components and biorecognition elements, HCPEs are useful for various sensing and imaging tasks.

Conjugation of P2 with anti-HER2 affibody was realized based on a carbodiimide-activated coupling reaction. Sodium dodecyl sulfate polyacrylamide gel electrophoresis (SDS-PAGE) confirmed the success of the coupling reaction. The anti-HER2—P2 conjugate ran at about 210 kDa, whereas pure anti-HER2 ran at about 7 kDa. Moreover, dynamic light scattering (DLS) indicated that the particle size of the anti-HER2 affibody-P2 conjugate was larger than pure P2, pure affibody and a P2/affibody mixture. Both SDS-PAGE and DLS confirmed the successful formation of affibody-attached P2.

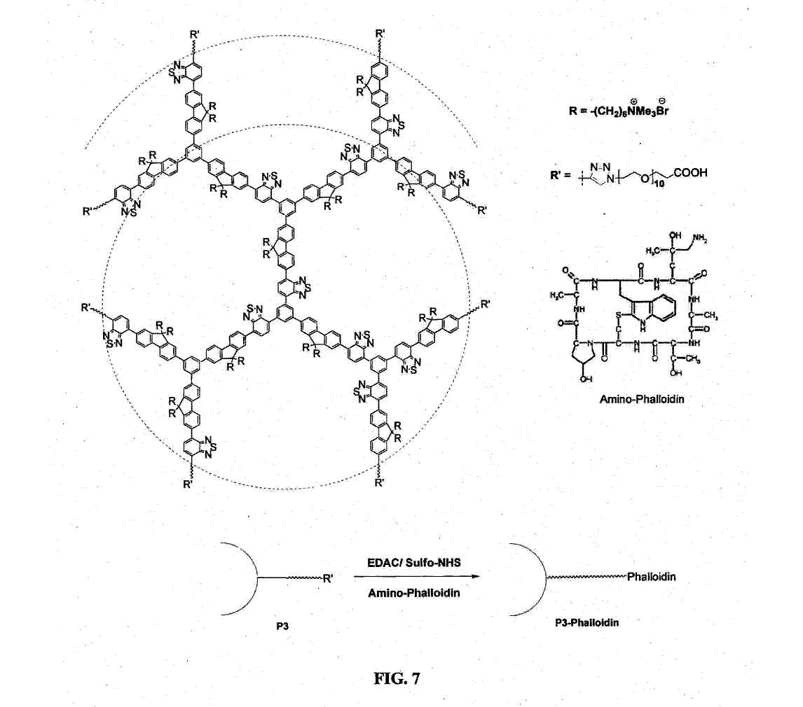

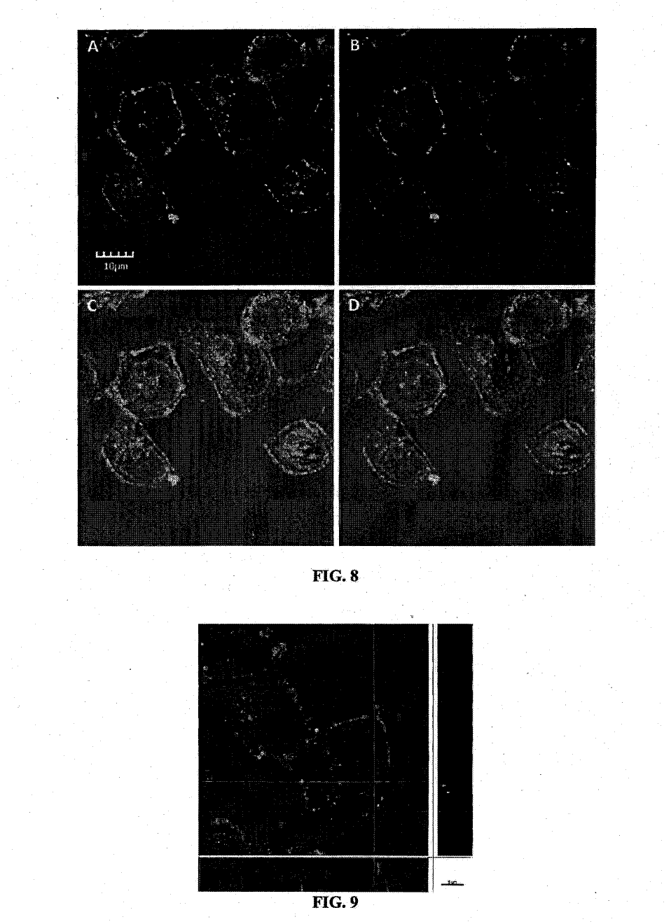

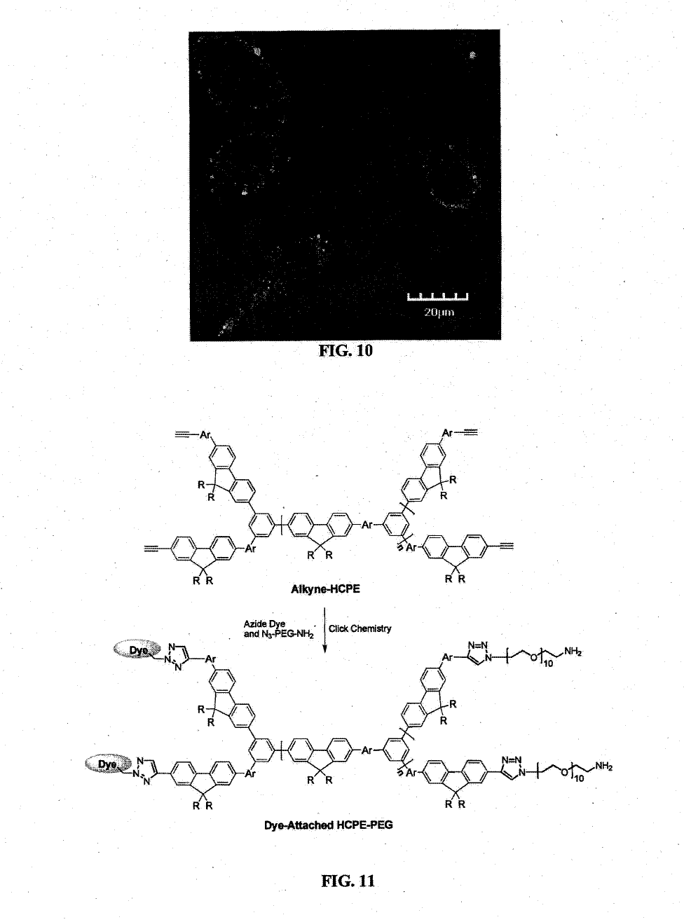

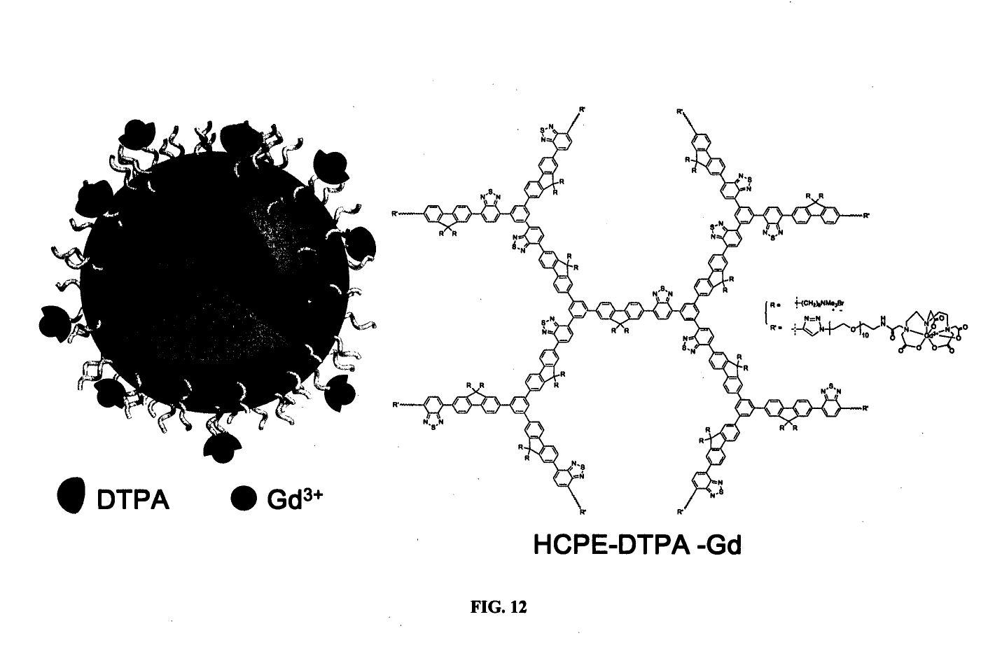

Conjugation of a COOH-terminated HCPE with phalloidin allowed specific staining of filamentous actin (F-actin) in living cells, specifically HeLa cells (FIGS. 9 and 10). Actin, an important protein in eukaryotic cells, is implicated in a number of cellular activities, including shape determination, cytokinesis, cell motility, and establishment of cell-cell and cell-matrix interactions. To achieve living cell imaging, the current commercial probes require microinjection or electroporation techniques to deliver the fluorescent probes into cells, which increase the complexity of the experiment and may disrupt the plasma membrane. Green fluorescent protein (GFP)-tagged protein can be integrated into actin filaments using transfection, a technique which provides an alternative way to observe filament dynamics in living cells. However, the transfection operation is always sophisticated and photobleaching of GFP limits its applications to long-term monitoring. The designed HCPE-conjugated phalloidin (FIG. 7) shows good living cell permeability and excellent photostability (FIG. 8).

The synthesis of P2 is depicted in FIG. 1.

### Synthesis of 4-(9,9′-Bis(6-bromohexyl)fluorenyl)-7-bromobenzothiadiazole (2)

2-(9,9-Bis(6-bromohexyl)fluorenyl)-4,4,5,5-tetramethyl-1,3,2-dioxaborolane (1) (2.84 g, 4.60 mmol), 4,7-dibromobenzothiadiazole (2.16 g, 7.36 mmol), Pd(PPh₃)₄ (53 mg, 0.046 mmol), potassium carbonate (4.43 g, 32.0 mmol) were placed in a 100 mL round bottomed flask. A mixture of water (12 mL) and toluene (30 mL) were added to the flask and the reaction vessel was degassed. The mixture was vigorously stirred at 90° C. for 2 days. After it was cooled to room temperature, dichloromethane was added to the reaction mixture. The organic portion was separated and washed with brine before drying over anhydrous MgSO₄. The solvent was evaporated off, and the solid residues were purified by column chromatography on silica gel using dichloromethane/hexane (1:5) as eluent to afford 2 as grassy yellow liquid (2 g, 62%). ¹H NMR (500 MHz, CD₃Cl, δ ppm): 8.0-7.87 (m, 3H), 7.85 (d, 1H, J=7.84 Hz), 7.77 (d, 1H, J=7.26 Hz), 7.66 (d, 1H, J=7.57 Hz), 7.45-7.30 (m, 3H), 3.27 (t, 4H, J=6.84 Hz), 2.14-1.97 (m, 4H), 1.74-1.62 (m, 4H), 1.32-1.18 (m, 4H), 1.17-1.04 (m, 4H), 0.83-0.66 (m, 4H). ¹³C NMR (125 MHz, CD₃Cl, δ ppm): 154.00, 153.35, 152.83, 150.90, 141.76, 140.50, 135.37, 134.49, 132.31, 128.24, 128.05, 127.58, 127.08, 123.79, 122.91, 120.13, 119.89, 112.81, 55.16, 40.12, 33.92, 32.60, 29.04, 27.73, 23.61. MS (MALDI-TOF): m/z 707.37 [M]⁺.

### Synthesis of 4-Bromo-7-(7-bromo-9,9′-bis(6-bromohexyl)fluorenyl)benzothiadiazole (3)

2 (0.80 g, 1.14 mmol) was dissolved in dichloromethane (20 mL) and cooled in an ice bath. Bromine liquid (0.45 g, 2.72 mmol) was then added slowly. After stirring at 45° C. for 12 hours, the reaction was quenched with sodium sulfite solution. Dichloromethane was added, and the organic portion was separated and washed with brine before drying over anhydrous MgSO₄. The solvent was evaporated, and the solid residues were purified by column chromatography on silica gel using dichloromethane/hexane (1:5) as eluent to afford 3 as yellow crystals (0.81 g, 90%). ¹H NMR (500 MHz, CD₃Cl, δ ppm): 7.95 (d, 1H, J=7.75 Hz), 7.91 (dd, 1H, J=1.33, 7.89 Hz), 7.88 (s, 1H), 7.81 (d, 1H, J=7.88 Hz), 7.64 (dd, 2H, J=8.12, 13.86 Hz), 7.50 (m, 2H), 3.28 (t, 4H, J=6.70 Hz), 2.0 (m, 4H), 1.67 (m, 4H), 1.23 (m, 4H), 1.11 (m, 4H), 0.73 (td, 4H, J=7.74, 15.61 Hz). ¹³C NMR (125 MHz, CD₃Cl, δ ppm): 153.98, 153.14, 150.46, 140.60, 139.54, 135.86, 134.20, 132.29, 130.30, 128.46, 128.13, 126.23, 123.83, 121.50, 120.04, 113.04, 55.51, 40.05, 33.96, 32.61, 29.00, 27.74, 23.60. MS (MALDI-TOF): m/z 785.44 [M]⁺.

### Synthesis of 4-(9,9′-Bis(6-bromohexyl)-7-((trimethylsilyl)ethynyl)fluorenyl)-7-((trimethylsilyl)ethynyl)benzothiadiazole (4)

A solution of trimethylsilyl acetylene (1.08 g, 1.55 mL, 11.0 mmol, d=0.695 g/mL) in diisopropylamine ((iPr)₂NH) (20.0 mL) was slowly added to a solution of 3 (3.9 g, 5.0 mmol), (Ph₃P)₂PdCl₂ (0.175 g, 0.25 mmol), and CuI (0.047 g, 0.25 mmol) in (iPr)₂NH (50.0 mL) under nitrogen at room temperature. The reaction mixture was then stirred at 70° C. for 8 hours. The solvent was removed under reduced pressure, and the residue was chromatographed on silica gel using hexanes as eluent to give 4 (2.8 g, 65%) as yellow crystals. ¹H NMR (500 MHz, CD₃Cl, δ ppm): 7.94 (m, 2H), 7.87 (d, 1H, J=7.39 Hz), 7.81 (d, 1H, J=7.85 Hz), 7.73 (d, 1H, J=7.28 Hz), 7.69 (d, 1H, J=7.82 Hz), 7.50 (d, 1H, J=7.86 Hz), 7.47 (s, 1H), 3.26 (t, 4H, J=6.79 Hz), 2.00 (m, 4H), 1.66 (m, 4H), 1.21 (m, 4H), 1.09 (m, 4H), 0.70 (td, 4H, J=7.70, 15.16 Hz), 0.36 (s, 9H), 0.30 (s, 9H). ¹³C NMR (125 MHz, CD₃Cl, δ ppm): 155.41, 153.20, 151.10, 150.87, 141.01, 140.91, 136.19, 135.16, 133.82, 131.43, 128.51, 127.27, 126.27, 123.86, 123.85, 121.85, 120.23, 119.85, 115.58, 106.05, 101.84, 100.52, 94.46, 55.27, 40.09, 33.90, 32.64, 29.00, 27.76, 23.57, 0.10, 0.04. MS (MALDI-TOF): m/z 819.70 [M]⁺.

### Synthesis of 4-(9,9′-Bis(6-bromohexyl)-7-ethynylfluorenyl)-7-ethynylbenzothiadiazole (5)

A KOH aqueous solution (3.0 mL, 20.0%) was diluted with methanol (15.0 mL) and added to a stirred solution of 4 (2.1 g, 2.5 mmol) in THF (20.0 mL). The mixture was stirred at room temperature for 6 hours and extracted with hexanes. The organic fraction was washed with water and dried over sodium sulfate. The crude product was chromatographed on silica gel using hexanes as the eluent. Recrystallization of the product from methanol gave 5 (1.6 g, 92%) as yellow crystals. ¹H NMR (500 MHz, CD₃Cl, δ ppm): 7.98 (dd, 1H, J=1.47, 7.87 Hz), 7.94 (s, 1H), 7.91 (d, 1H, J=7.34 Hz), 7.84 (d, 1H, J=7.90 Hz), 7.76 (d, 1H, J=7.47 Hz), 7.72 (d, 1H, J=7.80 Hz), 7.53 (dd, 1H, J=1.10, 7.63 Hz), 7.50 (s, 1H), 3.64 (s, 1H), 3.27 (t, 1H, J=6.74, 6.74 Hz), 3.17 (s, 1H), 2.03 (m, 4H), 1.66 (m, 4H), 1.22 (m, 4H), 1.10 (m, 4H), 0.71 (td, 4H, J=7.72, 15.20 Hz). ¹³C NMR (125 MHz, CD₃Cl, δ ppm): 155.61, 153.16, 151.15, 150.97, 141.24, 140.97, 136.15, 135.69, 133.98, 131.46, 128.55, 127.25, 126.55, 123.91, 120.84, 120.31, 120.07, 114.48, 84.52, 83.70, 79.55, 77.47, 55.27, 40.06, 33.88, 32.61, 29.02, 27.75, 23.60. MS (MALDI-TOF): m/z 673.01 [M]⁺.

Synthesis of Neutral Hyperbranched Conjugated Polymer (P0).

A Schlenk tube charged with 5 (100 mg, 0.15 mmol) was degassed with three vacuum-nitrogen cycles. A solution of cyclopentadienylcobaltdicarbonyl (CpCo(CO)₂) in anhydrous toluene (1.5 mL, 0.01 M) was then added to the tube, and the system was further frozen, evacuated, and thawed three times to remove oxygen. The mixture was vigorously stirred at 65° C. under irradiation with a 200 W Hg lamp (operating at 100 V) placed close to the tube for 8 hours. After the mixture was cooled to room temperature, it was dropped into methanol (100 mL) through a cotton filter. The precipitate was collected and redissolved in tetrahydrofuran. The resultant solution was filtered through 0.22 μm filter, and poured into hexane to further precipitate the product. After dried in vacuum at 40° C., P0 was obtained as a brown powder (65 mg, 65%). ¹H NMR (500 MHz, CDCl₃, δ ppm): 8.50-7.30 (m, 8H), 7.20 (br, 1H), 3.67 (s, 0.20H), 3.30 (br, 4H), 3.20 (s, 0.20H), 2.0 (br, 4H), 1.70 (br, 4H), 1.42-1.06 (m, 8H), 0.77 (br, 4H). ¹³C NMR (125 MHz, CDCl₃, 6 ppm): 155.41, 154.34, 153.73, 153.06, 151.10, 150.97, 150.91, 150.08, 141.43, 140.50, 137.87, 134.02, 131.45, 129.04, 128.53, 128.23, 126.54, 125.30, 123.97, 120.68, 120.30, 119.98, 84.60, 83.30, 80.88, 77.92, 55.27, 40.10, 33.91, 32.64, 29.06, 27.77, 23.65. Mₙ=6700, M_{w}/Mₙ=1.8.

Synthesis of Cationic HCPE (P1).

Trimethylamine (2 mL) was added dropwise to a solution of P0 (50 mg) in THF (10 mL) at −78° C. The mixture was stirred for 12 hours, and then allowed to warm to room temperature. The precipitate was redissolved by the addition of methanol (8 mL). After the mixture was cooled to −78° C., additional trimethylamine (2 mL) was added, and the mixture was stirred at room temperature for 24 hours. After removal of the solvent, acetone was added to precipitate P1 as a brown powder (55 mg, 95%). ¹H NMR (500 MHz, CD₃OD, δ ppm): 8.77-7.35 (m, 9H), 3.63 (s, 0.20H), 3.28 (br, 4H), 3.05 (s, 18H), 2.05 (br, 4H), 1.58 (br, 4H), 1.20 (br, 8H), 0.77 (br, 4H). ¹³C NMR (125 MHz, CD₃OD, δ ppm): 155.49, 154.10, 150.97, 141.91, 141.37, 140.70, 138.15, 134.00, 133.43, 131.07, 130.22, 128.54, 128.32, 126.23, 125.97, 123.89, 121.27, 121.13, 119.92, 87.08, 80.08, 66.31, 55.21, 52.20, 39.52, 28.73, 25.38, 23.29, 22.17.

Synthesis of Core-Shell HCPE (P2).

P1 (30 mg, 0.05 mmoL alkyne) and N₃-PEG-NH₂ (140 mg, 0.25 mmoL) were dissolved in DMF (5 mL). The mixture was degassed, and then N,N,N′,N″,N′″-pentamethyldiethylenetriamine (PMDETA) (12 mg, 0.0825 mmoL) and CuBr (11.8 mg, 0.0825 mmoL) were added. After reaction at 65° C. under nitrogen for 24 hours, the reaction mixture was cooled to room temperature and filtered through a 0.22 μm syringe driven filter. The filtrate was precipitated into diethyl ether to give a red powder. The crude product was redissolved in water and further purified by dialysis against Milli-Q water using a 3.5 kDa molecular weight cutoff dialysis membrane for 3 days. After freeze-drying, P2 (45 mg, 78%) was obtained as brown fibers. ¹H NMR (500 MHz, d₆-DMSO, δ ppm): 8.60-7.05 (m, 10.8H), 4.56-3.40 (m, 145H), 3.00-2.65 (m, 8H), 2.47-1.70 (m, 22H), 1.66-0.78 (m, 12H), 0.56 (br, 4H).

Synthesis of P2-Affibody Conjugate.

EDC aqueous solution (5 μL, 0.1 M) and P2 (2 μL, 1 mM) was added into borate buffer (150 μL, 10 mM). Then, sulfo-NHS (10 μL, 0.1 M) was added to the solution. After 1 hour, the solution was passed through a NapTM-5 column (Sephadex G-25, GE Healthcare) with water as an eluent to remove unreacted EDC and sulfo-NHS. To the eluted solution, an aqueous solution of anti-HER2 affibody (20 μL, 0.14 mM) was added under gentle stirring, and the solution was incubated for 1 hour at room temperature. Before cellular imaging experiments, the product was purified against 10 mM PBS using a 6.5 kDa molecular weight cutoff dialysis membrane for 2 hours.

The synthesis of phalloidin-P3 conjugate is depicted in FIG. 7.

Synthesis of HCPE-COOH (P3).

P1(30 mg, 0.05 mmoL alkyne) and N₃-PEG-COOH (142 mg, 0.25 mmoL) were dissolved in DMF (5 mL). The mixture was degassed, and then PMDETA (12 mg, 0.0825 mmoL) and CuBr (11.8 mg, 0.0825 mmoL) were added. After reaction at 65° C. under nitrogen for 24 h, the reaction mixture was cooled to room temperature and filtered through 0.22 μm syringe driven filter. The filtrate was precipitated into diethyl ether to give red powders. The crude product was redissolved in water and further purified by dialysis against Mill-Q water using a 3.5 kDa molecular weight cutoff dialysis membrane for 3 days. After freeze-drying, P3 (42 mg, 75%) was obtained as brown fibers. ¹H NMR (500 MHz, d₆-DMSO, δ ppm): 8.62-7.03 (m, 10.8H), 4.58-3.42 (m, 146H), 3.00-2.66 (m, 8H), 2.48-1.72 (m, 22H), 1.67-0.79 (m, 12H), 0.56 (br, 4H).

Synthesis of Phalloidin-P3 Conjugate.

The conjugation of HCPE-COOH (P3) with amino-phalloidin was carried out through EDAC coupling reaction. In brief, 0.11 mL of HCPE-COOH (18 mg/mL) aqueous solution and 0.38 mL of amino-phalloidin (1 mg/mL, methanol) were mixed and diluted to a total volume of 2 mL in borate buffer (0.2 M, PH=8.5). EDAC and Sulfo-NHS were then added into the solution at the stoichiometric molar ratio of HCPE-COOH/EDAC/Sulfo-NHS=1/5/5. The reaction was gently mixed for 3 hours at room temperature. The obtained solution was dialyzed against MilliQ water for 48 hours and HCPE-phalloidin was collected after freeze-drying.

### Example 2

### Synthesis of a Folid Acid-Functionalized Molecular Brush

The molecular brush (P4.1) was synthesized via a stepwise “grafting onto” method involving click chemistry. P4.1 formed core-shell spherical nanoparticles in aqueous solution, wherein the PEG grafting chains constituted the shell layer encapsulating the charged, conjugated backbones. Such a self-assembled nanostructure not only resulted in a high PL quantum yield in aqueous solution (11%), but also led to minimal nonspecific interactions with biomolecules and suppressed nonspecific cellular uptake. These desirable biochemical and optical properties make P4.1 an effective FR/NIR cellular probe for discrimination and visualization of MCF-7 cancer cells from NIH-3T3 normal cells in a high contrast and selective manner. In view of its high photostability and low cytotoxicity, such a molecular brush based cellular nanoprobe holds great promises as an alternative to current stains such as QDs and silica nanoparticles for clinical diagnosis and modern biological research.

In terms of materials design, the click-chemistry based “grafting onto” approach has the feasibility and flexibility to vary the components of both grafting chains and conjugated backbones for the control of water-solubility, self-assembly and optical properties of CPE-based molecular brushes. For instance, changing the BT units of P4.1 into 4,7-di(thien-5′-yl)-2,1,3-benzothiadiazole can further red-shift the emission maximum. From the application perspective, the presence of bio-amenable functional groups of CPE-based molecular brushes allows for facile attachment of different bio-recognition elements (such as antibodies, aptamers and peptides) to fulfill various sensing and imaging tasks.

Molecular brushes are unique macromolecules with densely grafted side chains on a linear polymeric backbone. Although several “grafting from” methods including nitroxyl radical mediated polymerization (NRMP) and atom transfer radical polymerization (ATRP) have been utilized to synthesize neutral conjugated polymer-based molecular brushes, the resulting polymers cannot be further functionalized. In comparison, a “grafting onto” strategy is more versatile as it offers a facile way to modify the brush prior to attachment onto the backbone, while the brush density is strongly limited by the grafting chemical reaction used. The Huisgen 1,3-dipolar cycloaddition reaction between organic azides and alkynes, known as click chemistry allows post-polymerization functionalization with nearly quantitative yield, mild reaction conditions, and broad functional group compatibility. In light of these considerations, the “grafting onto” strategy based on click chemistry was adopted to synthesize the surface-amenable CPE-g-PEG molecular brush.

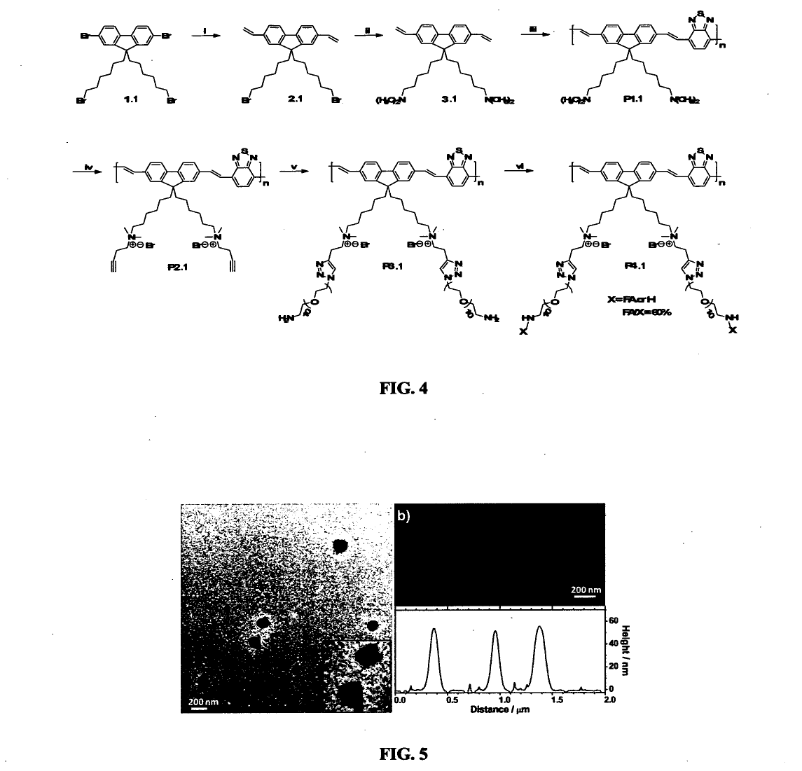

The synthetic route toward the CPE-g-PEG molecular brush and its FA-functionalized derivate is shown in FIG. 4. 9,9-Bis(6′-bromohexyl)-2,7-divinylfluorene (2.1), was synthesized in 78% yield by heating the mixture of 2,7-dibromo-9,9-bis(6′-bromohexyl)-fluorene (1.1) and tributylvinyltin in toluene at 100° C. for 24 hours using PdCl₂(PPh₃)₂/2,6-di-tert-butylphenol as catalyst. Treatment of 2.1 with dimethylamine in THF afforded the divinyl monomer, 9,9-bis(6′-(N,N-dimethylamino)hexyl)-2,7-divinylfluorene (3.1). After successful determination of the chemical structure of 3.1 by NMR and mass spectrometry, it was polymerized with 4,7-dibromobenzothiadiazole (4.1) via a Pd(OAc)₂/P(o-tolyl)₃ catalyzed Heck coupling reaction in the mixture of DMF/TEA (2:1) at 100° C. to afford the neutral polymer, poly[9,9-bis(6′-(N,N-dimethylamino)hexyl))fluorenyldivinylene-alt-4,7-(2′,1′,3′,-benzothiadiazole) dibromide] (P1.1). Quaternization of P1.1 with 4-bromobut-1-yne in the mixture of THF/DMF/DMSO at 55° C. gave the clickable cationic polymer, poly[9,9-bis(N-(but-3′-ynyl)-N,N-dimethylamino)hexyl))fluorenyldivinylene-alt-4,7-(2′,1′,3′,-benzothiadiazole) dibromide] (P2.1). This polymer precursor has alkyne groups at the end of the side chains, which allows for subsequent click reaction with azide compounds. The click reaction was carried out in DMF between P2.1 and azide-functionalized monodispersed PEG-NH₂ (N₃-PEG-NH₂) at 65° C. using N,N,N′,N″,N′″-pentamethyldiethylenetriamine (PMDETA) and CuBr as the catalyst, leading to the CPE-g-PEG (P3.1). Finally, coupling reaction between the amine groups of P3.1 and γ-carboxylic acid of FA using dicyclohexylcarbodiimide (DCC) and N-hydroxysuccinimide (NHS) as the catalyst in DMSO gave the FA-functionalized CPE-g-PEG (P4.1). The cationic polymers P2.1, P3.1 and P4.1 were purified by micro-filtration, precipitation, and finally dialysis against Milli-Q water using a 3.5 kDa molecular weight cutoff dialysis membrane for 3 days.

The chemical structures of these polymers were determined by ¹H NMR spectra. As compared to P1.1, a new peak at 3.08 ppm appears in the ¹H NMR spectrum of P2.1, which is assigned to the alkyne protons. The integral ratio of the peak at 3.08 ppm to that at 2.64 ppm (corresponding to the methylene protons near the 9-position of fluorene) is close to 0.48, indicating that the degree of quaternization is ˜96%. The successful click reaction is verified by the presence of a single resonance peak at 8.00 ppm in the ¹H NMR spectrum of P3.1, which corresponds to the proton next to the nitrogen atom of the triazole group. Comparison of the integrated areas between the multiple peaks ranging from 4.56 to 3.40 ppm (assigned to the methylene protons of PEG) and the peak at 0.56 ppm (assigned to the methylene protons secondly close to the 9-position of fluorene) reveals a high PEG graft efficiency of ˜90%, which is attributed to the high activity of the click reaction using PMDETA/CuBr as the catalyst. After FA functionalization of P3.1, the ¹H NMR spectrum becomes more complicated for P4.1. Nevertheless, the characteristic proton resonance peak of FA located at 8.66 ppm is separated from those of the conjugated backbone. Thereby, the molar percentage of FA in P4.1 is calculated to be −60%.

### Synthesis of 9,9-Bis(6′-(N,N-dimethylamino)hexyl)-2,7-divinylfluorene (3.1)

Dimethylamine solution (5 mL, 5.6 M in absolute ethanol) was added dropwise to a solution of 2.1 (500 mg, 0.92 mmol) in THF (8 mL) at room temperature. After stirring for 12 hours, additional dimethylamine solution (3 mL) was added, and the mixture was stirred at room temperature for 12 hours. The solvent was then removed under reduced pressure, and the residue was washed with hexanes and methanol to afford 3.1 (370 mg, 85%) as a white powder. ¹H NMR (500 MHz, CDCl₃, δ ppm): 7.61 (d, 2H, J=7.78 Hz), 7.39 (d, 2H, J=7.12 Hz), 7.35 (s, 2H), 6.79 (dd, 2H, J=10.85, 17.54 Hz), 5.82 (d, 2H, J=17.54 Hz), 5.27 (d, 2H, J=10.85 Hz), 2.14 (s, 12H), 2.10 (m, 4H), 1.96 (m, 4H), 1.27 (m, 4H), 1.08 (m, 8H), 0.65 (m, 4H). ¹³C NMR (125 MHz, CDCl₃, δ ppm): 151.25, 140.72, 137.40, 136.51, 125.30, 120.48, 119.72, 113.04, 59.79, 54.88, 45.46, 40.33, 29.90, 27.59, 27.07, 23.68. EIMS (m/z): 472.30 (M⁺).

### Synthesis of Poly[9,9-bis(6′-(N,N-dimethylamino)hexyl))fluorenyldivinylene-alt-4,7-(2′,1′,3′,-benzothiadiazole)dibromide] (P1.1)

A Schlenk tube was charged with 3.1 (100 mg, 0.212 mmol), 4.1 (62 mg, 0.212 mmol), Pd(OAc)₂ (2 mg, 9 μmmol), and P(o-tolyl)₃ (15 mg, 49 μmol) before it was sealed with a rubber septum.

The Schlenk tube was degassed with three vacuum-argon cycles to remove air. Then, DMF (1.6 mL) and triethylamine (0.8 mL) were added to the Schlenk tube and the mixture was frozen, evacuated, and thawed three times to remove air. The polymerization was carried out at 100° C. under vigorous stirring for 12 hours. It was then filtered through a 0.22 μm syringe driven filter and the filtrate was poured into diethyl ether. The precipitate was collected and washed with methanol and acetone, and then dried under vacuum for 24 hours to afford P1.1 (108 mg, 81%) as red fibers. ¹H NMR (500 MHz, CDCl₃, δ ppm): 8.14 (br 4H), 7.93-7.36 (m, 8H), 2.30 (br, 4H), 2.13 (s, 12H), 2.00 (br, 4H), 1.30 (br, 4H), 1.12 (br, 8H), 0.73 (br, 4H). ¹³C NMR (125 MHz, CDCl₃, δ ppm): 154.05, 151.68, 141.21, 136.71, 133.89, 129.43, 127.04, 126.43, 123.92, 121.25, 120.18, 59.77, 55.17, 45.41, 40.51, 29.98, 27.59, 27.17, 23.82. Mₙ=9500, M_{w}/Mₙ=2.1.

### Synthesis of Poly[9,9-bis(N-(but-3′-ynyl)-N,N-dimethylamino)hexyl))fluorenyldivinylene-alt-4,7-(2′,1′,3′,-benzothiadiazole) dibromide] (P2.1)

4-Bromobut-1-yne (2 mL) was added to P1.1 (50 mg) in THF (5 mL) and DMF (5 mL), and the mixture was stirred at 55° C. for 2 hours. Then, DMSO (5 mL) was added to dissolve the precipitate. After reaction for 48 hours, THF and methanol were removed under reduced pressure. The residue was then poured into acetone to give the crude product as a dark red powder. The product was further purified by dialysis against Milli-Q water using a 3.5 kDa molecular weight cutoff dialysis membrane for 3 days. After freeze-drying, P2.1 (56 mg, 78%) was obtained as red fibers. NMR (500 MHz, d₇-DMF, δ ppm): 8.53 (br, 4H), 8.36-8.18 (m, 6H), 7.99 (br, 2H), 3.65 (br, 3.84H), 3.42 (br, 4H), 3.20 (br, 3.84H), 3.08 (t, 1.92H), 2.95 (br, 12H), 2.44 (br, 4H), 1.79 (br, 4H), 1.32 (br, 8H), 0.88 (br, 4H).

Synthesis of PFVBT-g-PFG (P3.1):

P2.1 (30 mg, 0.05 mmoL alkyne) and N₃-PEG-NH₂ (140 mg, 0.25 mmoL) were dissolved in DMF (5 mL). The mixture was degassed, and then N,N,N′,N″,N′″-pentamethyldiethylenetriamine (PMDETA) (12 mg, 0.0825 mmoL) and CuBr (11.8 mg, 0.0825 mmoL) were added. After reaction at 65° C. under nitrogen for 24 hours, the reaction mixture was cooled to room temperature and filtered through a 0.22 μm syringe driven filter. The filtrate was precipitated into diethyl ether to give a red powder. The crude product was redissolved in water and further purified by dialysis against Milli-Q water using a 3.5 kDa molecular weight cutoff dialysis membrane for 3 days. After freeze-drying, P3.1 (45 mg, 78%) was obtained as a red powder. ¹H NMR (500 MHz, d₆-DMSO, δ ppm): 8.60-7.05 (m, 10.8H), 4.56-3.40 (m, ˜145H), 3.00-2.65 (m, 8H), 2.47-1.70 (m, ˜22H), 1.66-0.78 (m, 12H), 0.56 (br, 4H).

Synthesis of PFVBT-g-PEG-FA (P4.1):

The carboxylic acid group of FA (16.5 mg, 0.0335 mmol) dissolved in DMSO (0.8 mL) was pre-activated with DCC (8.25 mg, 0.04 mmol) and NHS (7.5 mg, 0.065 mmol) at room temperature. In the reaction, dicyclohexylurea was formed and removed by filtration. Although FA has α- and γ-carboxylic acid groups, γ-carboxylic acid was primarily activated in the DCC/NHS reaction due to its higher reactivity. P3.1 (12 mg, 0.02 mmoL —NH₂) was added to the NHS-activated FA solution. The reaction was kept at room temperature for 48 hours. The product was further purified by dialysis against Milli-Q water using a 3.5 kDa molecular weight cutoff dialysis membrane for 3 days. After freeze-drying, P4.1 (22 mg, 72%) was obtained as a red powder. ¹H NMR (500 MHz, d₆-DMSO, δ ppm): 8.66 (s, 1.2), 8.13-6.56 (m, 13H), 5.57 (br, 2.4), 4.50-2.60 (m, 157H), 2.3-1.44 (m, 27H), 1.36-0.93 (m, 12H), 0.76 (br, 4H).

### Example 3

### Self-Assembly Properties of P2

High-resolution transmission electron microscopy (HR-TEM) shows that P2 self-assembles into spherical nanoparticles with an average diameter of 30 nm in aqueous solution. Moreover, these nanospheres possess a core-shell nanostructure, wherein the dark interior and the gray exterior correspond to the domains enriched with electron-rich conjugated segments and saturated PEG chains, respectively. Such a core-shell nanostructure is beneficial to both bioconjugation and cell imaging, as PEG shells could serve as a protective layer.

### Example 4

### Self-Assembly Properties of P4.1

The self-assembly behaviors of P3.1 and P4.1 in water were at a concentration of 2 μm based on repeat unit (RU) studied by laser light scattering (LLS). Unimodal distribution peak was observed for both polymer solutions, revealing the formation of micellar nanoparticles with mean diameters of 108 and 135 nm for P3.1 and P4.1, respectively. Moreover, the narrow polydispersity of P3.1 (0.17) and P4.1 (0.22) indicates that the assembled nanoparticles are uniform in size. The larger particle size of P4.1 as compared to that of P3 should be ascribed to the additional FA groups on the side chains of P4.1.

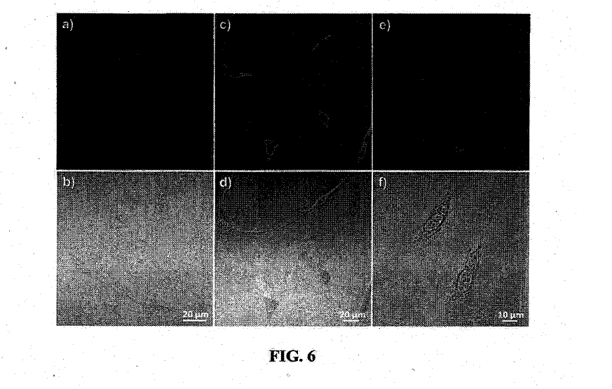

The morphology of P4.1-assembled nanoparticles in the dry state was further investigated by transmission electron microscopy (TEM) and tapping-mode atomic force microscopy (AFM) after depositing onto copper grid and mica, respectively. As shown in FIG. 6a, spherical nanoparticles with an average diameter of 150 nm are observed by TEM. Moreover, the inner part of the nanoparticles is darker than the outer part (Inset of FIG. 6a), manifesting a core-shell micelle structure. Considering the high electron density of the unsaturated π-conjugated backbone of P4.1, the inner core and outer shell of the nanoparticles should be enriched with the conjugated backbones and PEG grafting chains, respectively. The AFM image in FIG. 6b also shows the spherical morphology of P4.1-assembled nanoparticles with an average diameter of 155 nm. Furthermore, the cross-sectional analysis of the AFM image illustrates that the vertical height of the nanoparticles is 55 nm, which is smaller than the diameter. The three-dimensional disparity indicates that the nanoparticles collapse upon transforming from solution state into dry state. This observation rationalizes the larger diameter measured using TEM and AFM as compared to that using LLS. It is anticipated that the PEG-encapsulated micellar nanostructure of P4.1 in aqueous solution should be beneficial to resisting nonspecific interactions with charged biomolecules, facilitating specific cellular uptake.

### Example 5

### Optical Properties of P2

The absorbance and photoluminescence (PL) spectra of P1 and P2 in water were obtained. The absorption peaks of P2 are at 315 and 422 nm, corresponding to the fluorene and benzothiadiazole (BT) units, respectively. As compared to P1, the BT absorption peak of P2 is red-shifted by 12 nm, indicating the elongated effective conjugation length due to the generation of triazole units after click chemistry. The PL maximum of P2 is at 565 nm, which is blue-shifted by 33 nm relative to that of P1. In addition, the PL quantum yield of P2 in water is 0.12, which is higher than that of P1 (0.03). As previous reports have revealed that charge-transfer involved BT emission is sensitive to environmental polarity, these data indicate that the conjugated segments of P2 are localized in a microenvironment with lower polarity relative to that for P1, due to the formation of a PEG protective layer. With sufficiently high yellow fluorescence and an extremely large Stoke Shift (143 nm), P2 is suitable for fluorescence imaging.

To evaluate the nonspecific biological interactions of P1 and P2, the PL spectra of both polymer solutions upon addition of charged bovine serum albumin (BSA) were monitored. The fluorescence of P1 is significantly enhanced with increased [BSA], indicating that nonspecific interactions exist and induce the formation of complexes within which the local hydrophobicity of P1 decreases. In contrast, the fluorescence of P2 remains nearly the same upon addition of BSA, suggesting no significant interactions occurred between P2 and BSA owing to the shielding of PEG shells. These data prove that the core-shell molecular architecture can effectively prevent the conjugated segments of HCPE from interacting with biomolecules, thus allowing for efficient bioconjugation.

### Example 6

### Optical Properties of P2.1, P3.1 and P4.1

The optical properties of P2.1, P3.1, and P4.1 in water were studied and compared. The polymer concentration based on RU is 2 μm. P2.1 and P3.1 share two absorption maxima at 375 and 505 nm corresponding to the fluorene and BT units, respectively. P4.1 has two peaks centered at 282 and 505 nm with a shoulder at 375 nm. The new peak at 282 nm is ascribed to the FA absorption. According to the molar absorption coefficient of FA at 282 nm (˜2.5×10⁴ cm⁻¹ M⁻¹), the concentration of FA is estimated to be approximately 2.3 μm. Thus, the molar percentage of FA in P4.1 should be 57.5%, which coincides well with the ¹H NMR data (60%). The emission maximum of P2.1 is located at 673 nm, which is blue-shifted to 630 and 635 nm for P3.1 and P4.1, respectively. However, P3.1 and P4.1 have intense emission tails extending to 850 nm, allowing for FR/NIR fluorescence imaging. The PL quantum yields of P2.1, P3.1, and P4.1 are 1%, 12%, and 11%, respectively, measured using quinine sulfate in 0.1 M H₂SO₄ (η=55%) as the standard. As a result of the substantially stronger fluorescence of P3.1 and P4.1 as compared to that of P2.1, naked-eye visualization of the solution fluorescence of P3.1 and P4.1 becomes possible.

Our previous reports have revealed that the lowest unoccupied molecular orbital (LUMO) and highest occupied molecular orbital (HOMO) transition of poly(fluorene-alt-BT) derivatives is accompanied by charge transfer from the fluorene segments to the BT units due to strong electron deficiency of BT. This also occurs for P2.1, P3.1 and P4.1 due to their similar backbone structures. Accordingly, the low quantum yield of P2.1 in water originates from the charge transfer character of the excited states, which leads to quenched fluorescence. In contrast, the increased quantum yields and blue-shifted emission maxima of the PEG-grafted CPEs (P3.1 and P4.1) relative to P2.1 indicate that the PEG grafting chains provide a hydrophobic microenvironment for the conjugated backbones against water invasion, resulting in less quenched fluorescence in water. This is reasonable as the LLS, TEM and AFM data confirm that P3.1 and P4.1 form core-shell micellar nanoparticles in aqueous solution where the conjugated backbones are mainly localized in the core. Moreover, the dense bulky PEG grafting chains could inhibit intramolecuar and intermolecular π-π stacking to reduce the formation of low-emissive defects. This molecular effect should also partially contribute to the high quantum yields of P3.1 and P4.1, which are the highest among reported red-fluorescent water-soluble CPEs.

Due to the presence of a variety of biomolecules in both culture medium and cellular compartments, electrostatic and hydrophobic interactions between the cellular probe and biomolecules may exist to disturb the optical signals and cellular uptake. To evaluate the environmental stability of polymer fluorescence, BSA was added into the aqueous solution of P2.1, P3.1 and P4.1, and their fluorescence was monitored. BSA is chosen as the model biomolecule because it is abundant in culture medium, and has surfactant-like hydrophobic interactions with small fluorophores, and charged or neutral CPEs in aqueous media.

The PL spectra of P2.1, P3.1 and P4.1 at [RU]=2 μm in 25 mM phosphate buffered saline (PBS, pH=7.4) in the absence and presence of BSA were collected. Addition of BSA induces a progressive intensity increase in the emission of P2.1, concomitant with a gradual blue-shift of its emission maximum. The saturation occurs at [BSA]=0.25 μM. Since BSA has net negative charges (−17) at pH=7.4, the total net negative charges of BSA (4.25 μM) are nearly equal to the positive charges of P2.1 (4 μM) at the saturation point. The charge balance related saturation implies that the fluorescence response of P2.1 toward BSA is mainly controlled by electrostatic interactions. At the saturation point, the PL intensity of P2.1/BSA is approximately 19-fold higher than that of P2.1 alone, while the emission maximum is blue-shifted from 675 nm to 640 nm. Such a substantial spectral change of P2.1 upon addition of BSA is attributed to the increased hydrophobicity within the supramolecular complexes of P2.1/BSA. In contrast to P2.1, P3.1 and P4.1 show almost no fluorescence changes upon addition of BSA, suggesting no variation occurs for the local environment of the conjugated backbones owing to the core-shell nanostructures in aqueous solution. The difference in fluorescence responses of P2.1 versus P3.1 and P4 toward BSA indicates that grafting PEG onto the CPEs as the side chains can effectively encapsulate the charged conjugated backbones and in turn prevents them from contacting with biomolecules, ultimately leading to stable optical properties for CPEs.

### Example 7

### Cell Imaging with P2-Affibody Conjugate

Targeted fluorescence imaging of HER2-positive cancer cells using a P2-affibody conjugate was investigated. SKBR-3 breast cancer cells with high HER2 expression were chosen as the target, while MCF-7 breast cancer cells and NIH-3T3 fibroblast cells lacking HER2 were used as the negative controls. The confocal laser scanning microscopy (CLSM) images of these cells treated with affibody-attached P2 are shown in FIG. 2. The cellular nuclei are stained by propidium iodide (PI). Strong fluorescence mainly focused at the cellular membrane is observed for SKBR-3 cells, while randomly-distributed weak fluorescence is detected for both MCF-7 and NIH-3T3 cells. The fluorescence intensity decreased by less than 10% after continuous laser scanning for 15 minutes, indicating good photostability of the probe. These images not only affirm the existence of the specific binding between affibody-attached P2 and HER2 receptors in extracellular domains, but also highlight the effectiveness of affibody-attached P2 in discrimination of HER2-positive cancer cells from others. In light of the low cytotoxicity and high photostability of P2 this cellular probe holds great promise in practical applications.

### Example 8

### Cell Imaging with P3-Phalloidin Conjugate

F-Actin labeling in living Hela cells with P3-phalloidin conjugate was studied. The confocal image shows that the ruffling membrane of living Hela cells is specifically labeled by P3-phalloidin conjugate, taking advantage of the good membrane permeability of HCPE and specific interaction between phalloidin and F-actin. After 15 minutes of continuous excitation, the green fluorescence from P3-phalloidin conjugate shows no obvious decrease, indicating its excellent photostability (FIGS. 8-10).

### Example 9

### Cell Imaging with P4.1

Cell imaging based on P3.1 and P4.1 is investigated with confocal laser scanning microscopy (CLSM). Breast cancer cell (MCF-7) and fibroblast normal cell (NIH-3T3) are tested in order to demonstrate the utility of P4.1 in targeted cancer cell imaging. After incubating the cells with the polymer solution (1 μM) for 1 hour, the cells were fixed for fluorescence imaging studies. The excitation wavelength was fixed at 543 nm, and the fluorescent signals were collected at 650 nm. The CLSM images of P3.1-stained MCF-7 cells are shown in FIG. 6. A few fluorescent dots with low brightness are observed for the cells, which are discretely localized in the cellular cytoplasm. These images imply that P3.1 is inefficiently internalized by MCF-7 cells, presumably because the PEG shell of the P3.1-assembled nanostructure inhibits nonspecific cellular uptake. Similar imaging patterns were observed for P3.1-stained NIH-3T3 cells.

The CLSM images of P4.1-stained MCF-7 and NIH-3T3 cells are also displayed in FIG. 6. Strong fluorescence from the cellular cytoplasm were observed for MCF-7 cells, indicating that P4.1 is efficiently internalized by MCF-7 cells and accumulated in the cytoplasm. In contrast, weak fluorescence with small staining area was observed for NIH-3T3 cells and indicates limited cellular uptake. The cell-discrimination capability of P4.1 originates from the presence of FA groups which endows it with specific cancer cell internalization via receptor-mediated endocytosis. In addition, the PEG grafting segments between FA and the charged conjugated backbones of P4.1 blocks nonspecific cellular uptake, and thus enhances the discrepancy in the fluorescence images between cancer cells and normal cells. These data illustrate that P4.1 is an effective macromolecular probe for FR/NIR targeted cancer cell imaging with good cellular specificity and high fluorescence contrast.

### Example 10

### Cytotoxicity Assays with P2

MTT assays were performed to assess the metabolic activity of NIH-3T3 fibroblast. NIH-3T3 cells were seeded in 96-well plates (Costar, IL, USA) at an intensity of 2×10⁴ cells/mL. After 48 hours incubation, the medium was replaced by P2 solution at the concentration of 0.01, 0.02 or 0.06 mg/mL and the cells were then incubated for 8 and 24 hours, respectively. After the designated time intervals, the wells were washed twice with PBS buffer then freshly prepared MTT (100 μL, 0.5 mg/mL) solution in culture medium was added into each well. The MTT medium solution was carefully removed after 3 h incubation in the incubator. Isopropanol (100 μL) was then added into each well and the plates were gently shaken for 10 minutes at room temperature to dissolve all the precipitate. The absorbance of MTT at 570 nm was monitored by the microplate reader (Genios Tecan). Cell viability was expressed by the ratio of the absorbance of the cells incubated with P2 solution to that of the cells incubated with culture medium only. In all cases, >90% of the cells were viable after 24 hours, even at the highest concentration of P2 tested.

### Example 11

### Cytotoxicity Assays with P4.1

To explore the potential of the CPE-g-PEG molecular brush in long-term clinical applications, the photostability and cytotoxicity of P4.1 were investigated. Changes in the CLSM images of P4.1-stained MCF-7 cells under continuous laser scanning were monitored to evaluate the photostability of P4.1 in cells. The intensity decrease in the fluorescence image is approximately 8% after continuous laser scanning for 15 minutes. In comparison, most commercial dyes (such as fluorescein, rhodamine and Cy5) usually lose their fluorescence within 3 minutes under CLSM laser illumination. The sustained brightness indicates P4.1 has a relatively high photostability, even under harsh physiological conditions. The dense PEG-grafting chains of P4.1 act as a protective shell to shield the fluorescent conjugated backbones from intensive contact with the oxygen-rich environment outside.

The cytotoxicity of P4.1 was evaluated in mouse embryonic fibroblast cells (NIH-3T3) using MTT cell-viability assay after being cultured with P4.1 solutions at the concentration of 2, 10 or 50 μM for 24, 48 or 72 hours. Noteworthy is that the concentrations of these polymer solutions are much higher than that used for practical cell imaging (1 μM). The cell viabilities were >90% within the tested period, indicating the low cytotoxicity of P4.1. This result is consistent with the previous studies using conjugated polymers (such as polyaniline, polypyrrole, and polythiophene derivatives) as electroactive biomaterials for tissue engineering applications. The good cytocompatibility of P4.1 should also benefit from its PEG brushes that are intrinsically compatible with living systems.

### Example 12

### Synthesis of Other HCPEs

To adjust the absorption and emission wavelength of HCPEs, new monomers (5a, 4b and 3c) were designed and synthesized. 5a and 4b can lead to HCPE-PEG with cationic charges, while 3c can lead to negatively charged HCPE-PEG.

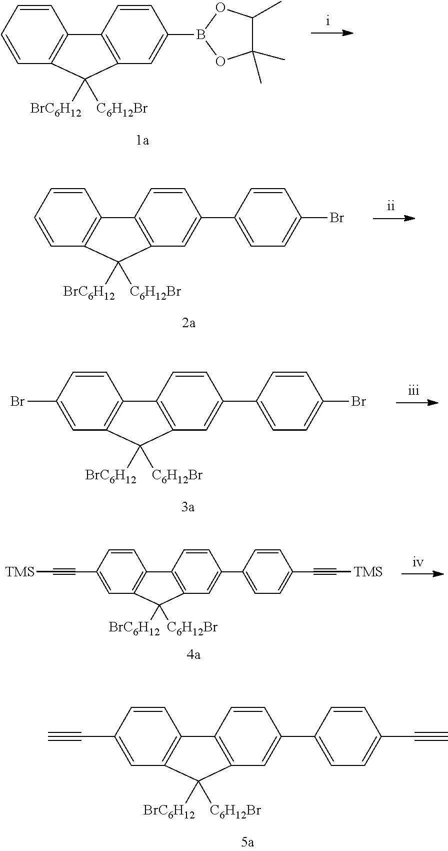

Synthesis of 2a.

2-(9,9′-Bis(6-bromohexyl)fluorenyl)-4,4,5,5-tetramethyl-1,3,2-dioxaborolane (1) (2.84 g, 4.60 mmol), 1,4-dibromobenzene (1.74 g, 7.36 mmol), Pd(PPh₃)₄ (53 mg, 0.046 mmol), potassium carbonate (4.43 g, 32.0 mmol) were placed in a 100 mL round bottomed flask. A mixture of water (12 mL) and toluene (30 mL) were added to the flask and the reaction vessel was degassed. The mixture was vigorously stirred at 90° C. for 2 days. After the mixture had cooled to room temperature, dichloromethane was added to the reaction mixture. The organic portion was separated and washed with brine before drying over anhydrous MgSO₄. The solvent was evaporated, and the solid residues were purified by column chromatography on silica gel using dichloromethane/hexane (1:5) as eluent to afford 2a.

Synthesis of 3a.

Compound 2a (1.14 mmol) was dissolved in dichloromethane (20 mL) and cooled in an ice bath. Bromine (2.72 mmol) was then added slowly.

After stirring at 45° C. for 12 hours, the reaction was quenched with sodium sulfite solution. Dichloromethane was added, and the organic portion was separated and washed with brine before drying over anhydrous MgSO₄. The solvent was evaporated, and the solid residues were purified by column chromatography on silica gel using dichloromethane/hexane (1:5) as eluent to afford 3a as yellow crystals (0.81 g, 90%).

Synthesis of 4a.

A solution of trimethylsilyl acetylene (1.08 g, 1.55 mL, 11.0 mmol) in diisopropylamine ((iPr)₂NH) (20.0 mL) was slowly added to a solution of 3a (5.0 mmol), (Ph₃P)₂PdCl₂ (0.175 g, 0.25 mmol), and CuI (0.047 g, 0.25 mmol) in (iPr)₂NH (50.0 mL) under nitrogen at room temperature. The reaction mixture was then stirred at 70° C. for 8 hours. The solvent was removed under reduced pressure, and the residue was chromatographed on silica gel using hexane as eluent to give 4a.

Synthesis of 5a.

An aqueous solution of potassium hydroxide (3.0 mL, 20.0%) was diluted with methanol (15.0 mL) and added to a stirred solution of 4a (2.1 g, 2.5 mmol) in THF (20.0 mL). The mixture was stirred at room temperature for 6 hours then was extracted with hexanes. The organic fraction was washed with water and dried over sodium sulfate. The crude product was chromatographed on silica gel using hexane as the eluent. Recrystallization of the product from methanol gave 5a (1.6 g, 92%) as yellow crystals.

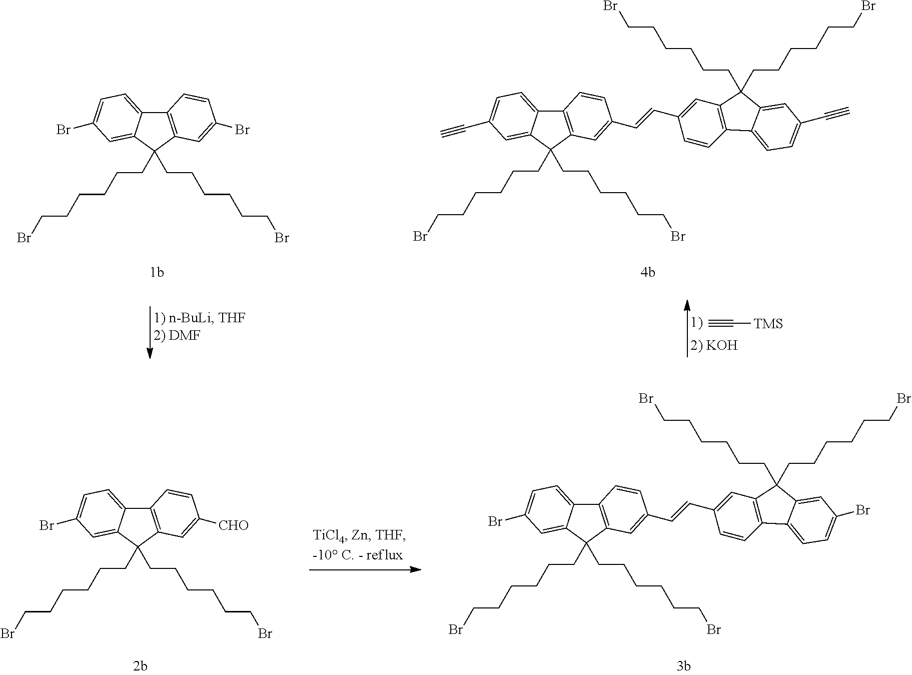

Synthesis of 2b.

A 25-mL round-bottomed flask was charged with 2,7-dibromo-9,9-di(6′-bromohexyl)fluorene (1.3 g, 2 mmol) in 20 mL of dry THF and cooled to −78° C. with a dry ice/acetone bath. At −78° C., n-BuLi in hexane (1.5 mL, 2.4 mmol) was injected and the mixture was stirred for 15 minutes. DMF (183 mg, 2.5 mmol) was added subsequently. The solution was stirred for 2 hours at −78° C. and then kept at room temperature overnight. The resulting mixture was quenched by water, and the solvent was removed by evaporation and extraction with chloroform. The organic phase was separated and dried over MgSO₄. After evaporation, the residue was purified with silica gel column chromatography (DCM/hexane=1:1) to yield 300 mg (25%) of 2b as a colorless oil.

Synthesis of 3b.

To a stirred suspension of zinc powder (0.5 g, 7.6 mmol) in dry THF (6 ml), was added TiCL₄ (0.4 ml, 3.6 mmol) slowly at −10° C. Then, a solution of 2-bromo7-formyl-9,9-di(6′-bromohexyl)fluorene (2b) (0.4 g, 0.7 mmol) in dry THF (2 ml) was added dropwise while the mixture was refluxed and stirred for 5 hours. The solution was quenched with saturated aqueous NaHCO₃ solution and extracted with ethyl acetate. The extract was washed with brine, dried over MgSO₄, and concentrated. The crude residue was purified by column chromatography using DCM/hexanes=1:3 to give 80 mg (68%) of 3b as colorless crystals.

Synthesis of 4b.

3b was then converted into 4b by firstly reacting with trimethylsilyl acetylene and then hydrolysis using KOH.

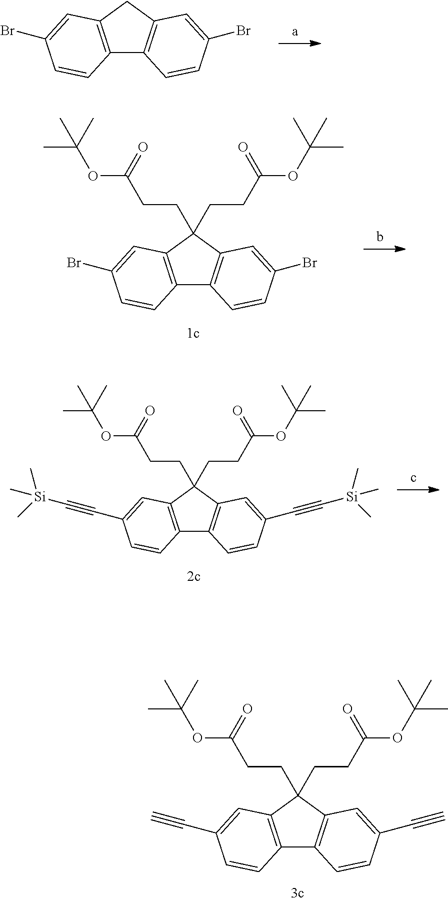

### Synthesis of 2,7-dibromo-9,9-bis(3-(tert-butyl propanoate))fluorene (1c)

To a solution of 2,7-dibromofluorene (3.3 g, 10.2 mmol) and tetrabutylammonium bromide (250 mg, 0.78 mmol) in toluene (25 mL) was added, a queous KOH (50 wt %, 5 mL) dropwise. The mixture was stirred at room temperature for 1 hour under argon atmosphere. Then, tert-butyl acrylate (5.25 g, 41 mmol) was added dropwise. 10 hours later, the mixture was diluted with dichloromethane and washed with water several times, then dried over MgSO₄. After removal of the solvents, the crude product was purified by silica gel column chromatography using a mixture of hexane/dichloromethane (6/4) as eluent, affording 1c as a white solid. Yield: 70%.

### Synthesis of 9,9-bis(3-(tert-butyl propanoate))-2,7-bis((trimethylsilyl)ethynyl)-fluorene (2c)

To a solution of 2,7-dibromo-9,9-bis(3-(tert-butyl propanoate))fluorene (2.9 g, 5 mmol), Pd(PPh₃)₂Cl₂ (175 mg, 0.25 mmol) and CuI (47 mg, 0.25 mmol) in (ⁱPr)₂NH (50 mL) under argon atmosphere, trimethylsilyl acetylene (1.96 g, 20 mmol) was added slowly. The reaction was stirred at 70° C. overnight. After removal of the solvent, the residue was redissolved in dichloromethane and washed with water several times. The crude product was purified by silica gel column chromatography using a mixture of hexane/dichloromethane (1/1) as eluent, affording 2c as a white solid. Yield: 95%.

### Synthesis of 9,9-bis(3-(tert-butyl propanoate))-2,7-diethynylfluorene (3c)

9,9-Bis(3-(tert-butyl propanoate))-2,7-bis((trimethylsilyl)ethynyl)fluorene (2.2 g, 3.5 mmol) was dissolved in a mixture of THF (50 mL) and methanol (25 mL) under argon atmosphere. An aqueous KOH solution (6 mL, 20 wt %) was added slowly. The mixture was stirred at room temperature. 1.5 hours later, the solvents were removed and the residue was dissolved in dichloromethane, washed with water several times, and dried over MgSO₄. Upon filtration and concentration, the residue was chromatographed on silica gel using hexane/dichloromethane (1/1) as eluent to give 3c as white crystals. Yield: 90%.

Synthesis of Dye-Functionalized HCPE-PEG.

A synthetic route for the preparation of a dye-functionalized HCPE-PEG conjugate is shown in FIG. 11. HCPE (0.05 mmoL alkyne), N₃-PEG-NH₂ and N₃-Alexa Fluor® 555 (140 mg, 0.25 mmoL) were dissolved in the mixture of THF (3 mL) and (2 mL). The mixture was degassed, and then PMDETA (12 mg, 0.0825 mmoL) and CuBr (11.8 mg, 0.0825 mmoL) were added. After reaction at 65° C. under nitrogen for 24 hours, the reaction mixture was cooled to room temperature and filtered through a 0.22-μm syringe driven filter. The filtrate was precipitated from diethyl ether to give a red powder. The red powder was dissolved in dichloromethane and washed with water and brine. The solvent was removed in vacuum, and the residue was precipitated from diethyl ether twice. After drying under vacuum at 40° C. for 24 hours, dye-functionalized HCPE-PEG was obtained.

Any commercially available azide-modified dye can be used for this reaction. The obtained polymers emit the dye fluorescence through energy transfer from the HCPE framework. Thus, the absorption and emission of HCPE-PEG can be fine-tuned for various applications.

### Example 13

### Streptavidin-HCPE Conjugate

Preparation.

The conjugation of HCPE-COOH with streptavidin was carried out through EDAC coupling reaction. In brief, 0.26 mg of HCPE-COOH and 0.6 mg streptavidin were mixed in 1 mL of borate buffer (0.1 m, PH=8.5). EDAC and Sulfo-NHS were then added into the solution at a stoichiometric molar ratio of HCPE-COOH/EDAC/Sulfo-NHS=1/10/10. The reaction was allowed to carry on for 3 hours at room temperature with gentle mixing. The obtained product was dialyzed using a 10 w Da molecular weight cutoff dialysis membrane against MilliQ water for 2 day to eliminate the excess EDAC and Sulfo-NHS as well as streptavidin.

Labeling Cell Surface Marker Epithelial Cell Adhesion Molecule (EpCAM).

MCF-7 cells (50 w) in 200 μL labeling buffer were incubated sequentially with 2 μg/mL primary anti-human CD326 antibody, 2 μg/mL biotinylated secondary anti-mouse IgG and 2 nM HCPE-streptavidin for 30 minutes each at room temperature, followed by washing twice. The obtained cells were collected for flow cytometry study using pure MCF-7 cells without treatment as the control group.

### Example 14

### Bimodal HCPE

Recently, various multimodal nanoparticle imaging probes have been investigated. In particular, nanomaterials combining fluorescence and magnetic resonance imaging (MRI) have attracted increasing interest because they have the advantages of both the high sensitivity of the fluorescence phenomenon and the high spatial resolution of MRI. In our case, the animo groups bound by HCPE were used to react with diethylenetriaminepentaacetic (DPTA) dianhydride, which is followed by labeling with gadolinium ions (Gd(III)), affording bimodal HCPE (Scheme 3). All HCPE-PEG with —COOH groups can be used for the synthesis of Gd(III)-labeled polymers. Other magnetic materials such as Mn²⁺ can also be used.

Synthesis of Gd(III)-Labeled HCPE.

5 mg of DTPA dianhydride (Aldrich) was added to a solution of HCPE-NH₂ (20 mg) in anhydrous DMSO (10 mL) containing 20 μL of triethylamine. The mixture was stirred at room temperature in the dark for 24 hours, which is followed by dialysis against MilliQ water using a 3.5 kDa molecular weight cutoff dialysis membrane for 2 days. Thereafter, excess amount of GdCl₃ was added to an aqueous solution of HCPE-DTPA for chelating. The mixture was maintained at pH 5.5 and allowed to react in the dark for 24 hours. The unbound Gd(III) was removed by dialysis against MilliQ water using a 3.5 kDa molecular weight cutoff dialysis membrane for 2 days. The obtained suspension was frozen and freeze-dried for 2 days to yield Gd(III)-labeled HCPE as a fine powder. The Gd(III) content in the product was determined by ion coupled plasma-mass spectrometry (ICP-MS, Agilent ICP-MS 7500).

In Vivo Fluorescence Imaging and MRI.

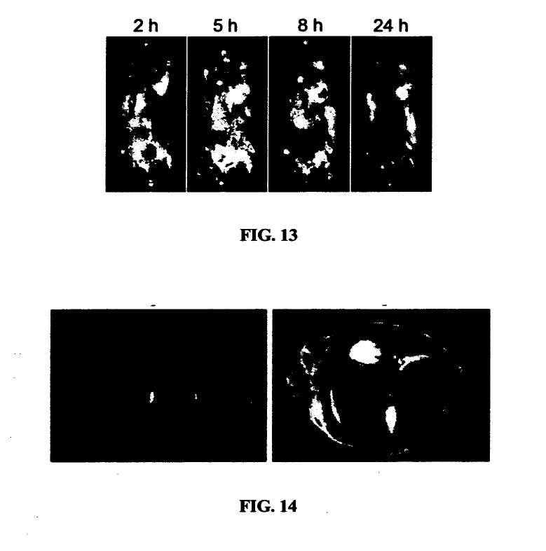

Male ICR mice implanted with murine hepatic carcinoma cell line H₂₂ were used to investigate the fluorescence imaging and MRI of Gd(III) labeled HCPE NPs. ICR mice were inoculated subcutaneously at the left axilla with Hₙ tumor cells (5-6×10⁶ cells per mouse). H₂₂ tumor-bearing mice were intravenously injected with 250 μl of Gd(III) labeled HCPE NPs at a dose of 20 μmol Gd/kg when the tumor volume reached a mean size of about 300 mm³. For in vivo fluorescence imaging, the mice were anesthetized and placed on an animal plate heated to 37° C. The time-dependent biodistribution in mice was imaged using the Maestro in vivo fluorescence imaging system (CRi Inc.). Light with a central wavelength at 455 nm was selected as the excitation source. In vivo spectral imaging from 500 to 900 nm at 10 nm steps was conducted with an exposure time of 150 milliseconds for each image frame. Autofluorescence was removed by using spectral unmixing software. Scans were carried out at 2 hours, 5 hours, 8 hours, and 24 hours post-injection. The results are shown in FIG. 13. Furthermore, MR imaging was also performed with a 7 T Micro-MR scanner (PharmaScan 70/17, Brucker). The instrumental parameters were set as follows: TR/TE=1300 ms/9 ms, field of view (FOV)=3.5×3.5 cm, matrix size=192×256, number of averages=1, and slice thickness=0.8 mm.

Cisplatin-Complexed HCPE NPs.

Theragnostic multifunctional nanoparticles have recently attracted increasing attention due to their abilities to achieve simultaneous diagnosis of disease, targeted drug delivery and drug tracking. In this case, cisplatin was complexed with HCPE NPs largely through complex formation between the platinum (II) of cisplatin and the terminal carboxylic group of PEG on the HCPE NP surface. Cisplatin (cis-dichlorodiammineplatinum (II)), one of the most widely used anticancer drugs, is very effective for the chemotherapy of a variety of solid tumors, such as gastrointestinal, genitourinary and liver cancers. However, the severely systemic side effects attributed to its poor blood circulation and significant accumulation in the kidneys greatly limit its clinical application. Hence, it is of high importance to prolong the blood circulation of cisplatin, improve its accumulation on target and trace its distribution in cells and in vivo.

Synthesis of Cisplatin-Functionalized HCPE NPs.

HCPE-PEG-COOH was dissolved in MilliQ water ([repeat unit (RU)]=1 mM), and then cisplatin was added at a molar ratio of 1:1 cisplatin:carboxylate groups of HCPE-PEG-COOH. The mixture was allowed to react at 37° C. for 3 days. The unbound cisplatin was removed by dialysis against MilliQ water using a 3.5 kDa molecular weight cutoff dialysis membrane for 3 days. The obtained suspension was frozen and freeze-dried for 2 days to yield cisplatin-functionalized HCPE NPs.

### Example 15

### Label-Free, Naked-Eye Detection of Lysozyme Using CPEs

Antilysozyme aptamer (5′-NH₂-ATC TAC GAA TTC ATC AGG GCT AAA GAG TGC AGA GTT ACT TAG SEQ ID NO.: 1) was ordered from Sigma-Genosys. Hen egg white lysozyme, BSA, and human trypsin were ordered from Sigma-Aldrich. Human R-thrombin was ordered from HTI.

Instrumentation.

The NMR spectra were collected on a Broker ACF400 (400 MHz). The absorption spectra of aptamer and lysozyme were measured using a UV-vis spectrometer (Shimadzu, UV-1700, Japan). The photoluminescence spectra were recorded on a fluorometer (Perkin-Elmer, LS-55) equipped with a xenon lamp excitation source and a Hamamatsu (Japan) 928 PMT, using 90° angle detection for solution samples. The size of silica NPs was calculated using a field emission scanning electron microscope (FE-SEM JEOLJSM-6700 F) after coating a thin Pt layer via a platinum coater. The zeta-potential of the NPs was measured using a zeta-potential analyzer (ZetaPlus, Brookhaven Instruments Corp.) at room temperature.

Synthesis and Characterization of PFVSO₃.

2,7-Dibromo-9,9-bis(2-(2-(2-methoxyethoxy)-ethoxy)ethyl)fluorene was synthesized according to our previous report. (See, for example, (a) Pu, K. Y., et al., Adv. Funct. Mater. 2008, 18, 1321-1328; (b) Wang, F. K.; Bazan, G. C., J. Am. Chem. Soc. 2006, 128, 15786-15792; (c) Pu, K. Y., et al., Chem. Mater. 2009, 21, 3816-3822, the entire teachings of which are incorporated herein by reference.)

### 9,9-Bis(2-(2-(2-methoxyethoxy)ethoxy)ethyl)-2,7-divinylfluorene (1)

2,7-dibromo-9,9-bis(2-(2-(2-methoxyethoxy)-ethoxy)ethyl)fluorene (1.23 g, 2.0 mmol), tributylvinyltin (1.33 g, 4.2 mmol), PdCl₂(PPh₃)₂ (56 mg, 0.09 mmol), 2,6-di-tert-butylphenol (8 mg, 38 mmol), and toluene (20 mL) were mixed in a 50-mL flask. The reaction mixture was stirred and heated at 100° C. for 24 hours under nitrogen. After cooling to room temperature, the mixture was diluted with ether, treated with an aqueous solution of HF (approximately 10%), and stirred for 12 hours. The mixed solution was then filtered to remove the solids, and the filtrate was dried over anhydrous MgSO₄. The solvent was removed under reduced pressure, and the residue was chromatographed on silica gel using hexanes/ethyl acetate (1:1) as eluent to give 1 (0.70 g, 68%) as a blue liquid. ¹H NMR (500 MHz, CDCl₃, δ ppm): 7.60 (d, 2H, J=7.8 Hz), 7.44 (s, 2H), 7.39 (d, 2H, J=7.7 Hz), 6.78 (dd, 2H, J=10.9 Hz, J=17.6 Hz), 5.80 (d, 2H, J=17.5 Hz), 5.27 (d, 2H, J=10.9 Hz), 3.51 (dd, 4H, J=3.4 Hz, J=5.9 Hz), 3.46 (dd, 4H, J=3.3 Hz, J=6.0 Hz), 3.39 (t, 4H, J=3.2 Hz), 3.33 (s, 6H), 3.21 (t, 4H, J=3.3 Hz), 2.76 (t, 4H, J=5 Hz), 2.40 (t, 4H, J=5.17 Hz). 13CNMR (125 MHz, CDCl₃, δ ppm): 149.50, 139.96, 137.00, 136.83, 125.82, 120.69, 119.85, 113.54, 71.83, 70.43, 70.39, 69.96, 66.98, 58.96, 50.96, 39.75.

### 2,7-Dibromo-9,9-bis(4-sulfonatobutyl)fluorene disodium (2)

2,7-Dibromofluorene (4 g, 12 mmol) and tetrabutylammoium bromide (80 mg) were dissolved in a mixture of a 50 wt % aqueous solution of sodium hydroxide (8 mL) and dimethyl sulfoxide (DMSO) (60 mL). A solution of 1,4-butane sultone (4 g, 29 mmol) in DMSO (20 mL) was added dropwise into the mixture under nitrogen. After stirring at room temperature for 4 hours, the reaction mixture was precipitated into acetone to afford the crude product. The product was collected by filtration, washed with ethanol, recrystallized twice from acetone/water, and dried under vacuum at 60° C. for 24 hours to yield 2 as white needle crystals (4.3 g, 58.6%). ¹H NMR (500 MHz, CD₃OD, δ ppm): 7.68 (d, J=8.11 Hz, 2H), 7.63 (d, 2H, J=1.45 Hz), 7.52 (dd, 2H, J=1.42, 8.08 Hz), 2.68-2.47 (m, 4H), 2.22-2.00 (m, 4H), 1.62 (td, 4H, J=7.83, J=7.83, J=15.65 Hz), 0.67 (td, 4H, J=7.83, J=7.83, J=15.65 Hz). ¹³C NMR (125 MHz, CD₃OD, δ ppm): 153.39, 140.68, 131.61, 127.38, 122.74, 122.52, 52.37, 40.76, 26.19, 24.25. MS (MALDI-TOF): m/z 619.89 [M−Na]. (See, for example, Huang, F., et al., Polymer 2005, 46, 12010-12015, the entire teachings of which are incorporated herein by reference.)

### Poly[9,9-bis(2-(2-(2-methoxyethoxy)ethoxy)ethyl)fluorenevinylene-alt-9,9-bis(4-sulfonatobutyl)fluorenevinylene Sodium Salt] (PFVSO₃)

1 (216 mg, 0.423 mmol), 2 (271 mg, 0.423 mmol), Pd(OAc)₂ (4.0 mg, 0.018 mmol), and P(o-tolyl)₃ (30 mg, 0.098 mmol) were placed in a round-bottomed flask. A mixture of DMF (3.0 mL), H₂O (1.0 mL), and triethylamine (1.5 mL) was added to the flask, and the reaction vessel was degassed. The mixture was vigorously stirred at 110° C. for 12 hours. The mixture was filtered through a 0.22 μm syringe driven filter unit, and the filtrate was poured into acetone. The precipitate was collected and washed with acetone and then dried under vacuum for 24 hours to afford PFVSO₃ (328 mg, 78%, Mn=15000) as yellow fibers. ¹H NMR (500 MHz, CD₃OD, δ ppm): 7.87-7.51 (m, 12H), 7.38 (br, 4 H), 3.54-3.39 (m, 12H), 3.36 (br, 4H), 3.27-3.13 (m, 6H), 2.90 (br, 4H), 2.57 (br, 8 H), 2.20 (br, 4H), 1.63 (br, 4H), 0.76 (br, 4H). ¹³C NMR (125 MHz, CD₃OD, δ ppm): 150.90, 149.97, 140.69, 140.00, 137.01, 128.63, 128.25, 126.13, 125.81, 120.86, 120.45, 119.71. 119.58, 71.45, 69.95, 69.91, 69.85, 69.82, 69.50, 57.74, 54.67, 51.18, 42.01, 39.20, 25.00.

Comparison of the integrated areas between the peak at 5.95 ppm and the peak at 0.76 ppm revealed that the number-average degree of polymerization (DP) of PFVSO₃ is approximately 15. Thus, the number-average molecular weight is approximately 15,000. The water solubility of PFVSO₃ is approximately 20 mg/mL at 24° C.

The absorbance and photoluminescence (PL) spectra of PFVSO₃ in water are depicted in FIG. 16. The polymer concentration based on repeat unit (RU) is 4 μM. PFVSO₃ has an absorption maximum at 428 nm and a shoulder peak at 455 nm, while its emission maximum is at 475 nm. While not wishing to be bound by any particular theory, the blue-green emission of PFVSO₃ is attributed to the introduction of CdC bond to the polymer backbone, which elongates the effective conjugated length relative to that of polyfluorene. The PL quantum yield of PFVSO₃ in water is 0.56 and was measured using quinine sulfate in 0.1M H₂SO₄ (quantum yield=0.55) as the reference. The high water solubility provided by the terminal sulfonate groups and the ethylene oxide side chains is thought to be responsible for the high quantum yield of PFVSO₃ in aqueous solution. (See Mikroyannidis, J. A.; Barberis, V. P. J. Polym. Sci., Part A: Polym. Chem. 2007, 45, 1481-1491.)

Preparation of Anti-Lysozyme Aptamer-Functionalized Silica NPs.

The bare silica NPs were synthesized according to a modified Stöber method, which yielded uniform NPs with a diameter of approximately 100 nm. (See Stöber, W., et al., J. Colloid Interface Sci. 1968, 26, 62-69, the entire teachings of which are incorporated herein by reference.) On the basis of the NP size and the density of silica (1.96 g cm⁻³), it can be estimated that 1.0 mg of the synthesized NPs contained approximately 1×10¹² NPs. Modification of the silica NP surface involved two steps. (See Wang, Y. S.; Liu, B. Anal. Chem. 2007, 79, 7214-7220, the entire teachings of which are incorporated herein by reference). First, the silica NP was reacted with 3-aminopropyltriethoxysilane (APTES) to generate amino groups on the NP surface. Then, the amino-functionalized NPs were treated with 2,4,6-trichloro-1,3,5-triazine to produce a triazine-covered surface for subsequent aptamer immobilization. After chemical modification, the triazine-functionalized silica NPs (1 mg) were dispersed in immobilization buffer (20.1 mM boric acid, 1.4 mM sodium tetraborate decahydrate, 1.2 M NaCl pH 8.5, 25 μL).

In heterogeneous assays, the kinetic and thermodynamic binding process of the analyte can be significantly influenced by the density of the recognition element on the solid support. (See, for example, (a) Peterson, A. W., et al., Nucleic Acids Res. 2001, 29, 5163-5168; (b) Gong, P.; Levicky, R., Proc. Natl. Acad. Sci. U.S.A. 2008, 105, 5301-5306; (c) Herne, T. M.; Tarlov, M. J., J. Am. Chem. Soc. 1997, 119, 8916-8920, the entire teachings of which are incorporated herein by reference.) Previous studies have shown that aptamer-target binding can be inhibited by densely-packed aptamers on gold rod electrodes due to cross-hybridization of individual aptamer sequences (See, for example, White, R. J., et al., Langmuir 2008, 24, 10513-10518, the entire teachings of which are incorporated herein by reference.)

To study the effect of aptamer density on lysozyme detection, different concentrations of aptamers, ranging from 2 to 36 μM were incubated with silica NPs (1 mg) to prepare Apt-NPs with different aptamer densities on the NP surface. The surface density, expressed as “number of aptamers per NP”, was determined by the ratio of the total number of immobilized aptamers to the total number of silica NPs in solution. Various aliquots of NH₂-aptamer solution (100 μM) from 0.5 to 9 μL were subsequently added into the NP suspension and incubated at room temperature for 14 hours. The NP suspension was centrifuged, and the supernatant was collected for absorbance measurements. The aptamer-immobilized NPs were washed with immobilization buffer. The number of immobilized aptamer molecules on the silica NPs was calculated from the absorbance difference at 260 nm between the aptamer solution before immobilization and the supernatant after immobilization and NP removal. The surface density was calculated to be in a range of 30 Apt/NP to 510 Apt/NP.

To minimize nonspecific absorption of proteins on NPs, ethanolamine was used to block the free triazine sites on the NP surface after aptamer immobilization. (See, for example, Wang, Y. S.; Liu, B. Chem. Commun. 2007, 34, 3553-3555; Frederix, F., et al., Biochem. Biophys. Methods 2004, 58, 67-74; the teachings of which are incorporated herein by reference.) Blocking was carried out by redispersing the Apt-NPs (1 mg) in blocking buffer (4 M ethanolamine, 20 mM Tris-HCl, 100 mM NaCl, 5 mM MgCl₂, pH=8.5, 200 μL) and incubating the resulting mixture for 1 hour at room temperature. The NP suspension was then centrifuged and washed with washing buffer (20 mM Tris-HCl, 100 mM NaCl, 5 mM MgCl₂, pH=8.5).

Optimization of Assay.

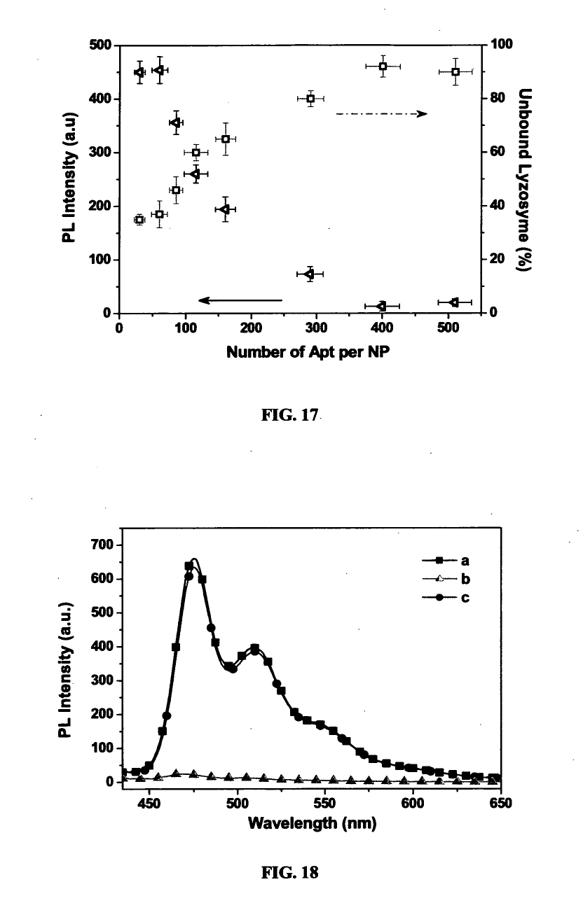

Aptamer-functionalized NPs (2 mg) with different probe densities were incubated with the same concentration of lysozyme (20 μg/mL), then washed. The lysozyme bound aptamer-NPs (lysozyme/Apt-NPs) were subsequently treated with 10 μM PFVSO₃ based on repeat unit (RU) for 5 minutes, which was followed by washing to remove excess polymer. The PL intensity of the final NP suspension was plotted as a function of aptamer surface density, and the results are shown in FIG. 17. The PL intensity significantly decreases with increased surface aptamer density, which could be ascribed to insufficient binding of lysozyme to aptamer at elevated surface density. (See Cheng, A. K. H., et al., Anal. Chem. 2007, 79, 5158-5164, the entire teachings of which are incorporated herein by reference). At low surface density, aptamers have more space which favors their G-quartet folding structure for lysozyme binding. However, in the case of high surface density, steric/conformational effects could hamper the specific binding between lysozyme and the aptamer. To further confirm this hypothesis, the adsorbed lysozyme was monitored according to the UV difference at 280 nm between the same lysozyme solution before incubation and the supernatant solution after incubation with different Apt-NPs and NP removal. As shown in FIG. 17, the percentage of unbound lysozyme increases with increased aptamer density on NPs, which verifies that more lysozyme molecules are captured by Apt-NPs at a low surface density. The optimum surface density was approximately 60 aptamers per NP (60 Apt-NP), where the polymer stained Apt-NP PL intensity reached the maximum, which is beneficial for effective lysozyme quantification.

To understand the surface charge change upon aptamer/lysozyme/PFVSO₃ interaction, the zeta-potentials of 60 Apt-NP, lysozyme/Apt-NPs (2 mg of 60 Apt-NP upon incubation with 20 μg/mL of lysozyme, followed by washing with washing buffer and redispersion), and PFVSO₃/lysozyme/Apt-NP (the obtained lysozyme/Apt-NPs upon further treatment with 1 μM PFVSO₃ followed by washing with water and redispersion) were measured. 60 Apt-NP possess a negative zeta-potential value of −39.34±1.55 mV, due to the large amount of negatively-charged aptamers on NP surface. The capture of lysozyme shifts the zeta potential from −39.35 to −14.96±0.88 mV, due to the presence of positively charged lysozyme molecules on NP surface. Staining with PFVSO₃ results in an increase in zeta-potential from −14.96 to −35.75±1.44 mV due to self-assembly between PFVSO₃ and lysozyme on NPs. This data confirms that the NP surface charge changes in the recognition event, which plays a vital role in lysozyme detection.

Lysozyme Detection Using Blocked Apt-NPs.

Various volumes of lysozyme (1.5 mg/mL) were added to the Apt-NPs (0.2 mg) in lysozyme reaction buffer (20 mM Tris-HCl, 100 mM NaCl, 5 mM MgCl₂, pH=8.5, 100 μL) to yield final lysozyme concentrations from 0 to 37.5 μg/mL. The resulting mixtures were incubated for 30 minutes at room temperature. Free lysozyme was removed and the NPs were washed with washing buffer three times. The lysozyme-associated NPs were redispersed in Milli-Q™ (Millipore Corp.) water (100 μL), and PFVSO₃ (100 μM, 1 μL) was added. The mixture was incubated for 5 minutes. Excess PFVSO₃ was washed away by a centrifugation-washing-redispersion process with washing buffer (100 mL, 3 times). The collected NPs were redispersed in 15 mM PBS buffer (pH=7.4) for fluorescence measurements.

Parallel experiments were conducted using a mixture of BSA (20 μg/mL), thrombin (20 μg/mL), and trypsin (20 μg/mL) to examine the assay specificity. BSA, human thrombin, and trypsin have pI values of 4.7, 7.0-7.6, and 10.5, respectively, with net negative, neutral, and positive charges on the protein surface under the experimental conditions. The 60 Apt-NP (0.2 mg) was incubated with lysozyme (20 μg/mL) as well as a mixture of interference proteins (20 μg/mL BSA, 20 μg/mL thrombin, and 20 μg/mL trypsin) in binding buffer (20 mM Tris-HCl, 100 mM NaCl, 5 mM MgCl₂, pH=8.5), followed by polymer staining ([RU]=1 μM) for 5 minutes and NP washing with washing buffer (20 mM Tris-HCl, 100 mM NaCl, 5 mM MgCl2, pH=8.5). The PL spectra of the redispersed NPs are shown in FIG. 18. Intense polymer emission at 475 nm is only witnessed in the presence of lysozyme due to the recognition-induced switching of lysozyme/Apt-NP charge, followed by PFVSO₃ self-assembly due to electrostatic interaction. No polymer fluorescence was observed in the presence of interference proteins. The nonspecific absorption of foreign proteins (e.g., positively charged trypsin) was largely avoided by blocking the NPs with ethaholamine and washing the NPs.

As such, PFVSO₃ hardly stains negatively charged Apt-NPs due to electrostatic repulsion in our experimental conditions and NPs remain nonfluorescent. In addition, the fluorescent signal from 60 Apt-NPs upon incubation with the mixture of lysozyme and interference proteins (20 μg/mL each) after washing is shown in curve c of FIG. 18. The polymer signal obtained from lysozyme in protein mixtures is almost the same as that from the pure lysozyme. The specific recognition of lysozyme in protein mixtures not only indicates the effectiveness of aptamer-protein binding but also highlights the intelligent target capture and interference isolation of the silica NP sensing platform.

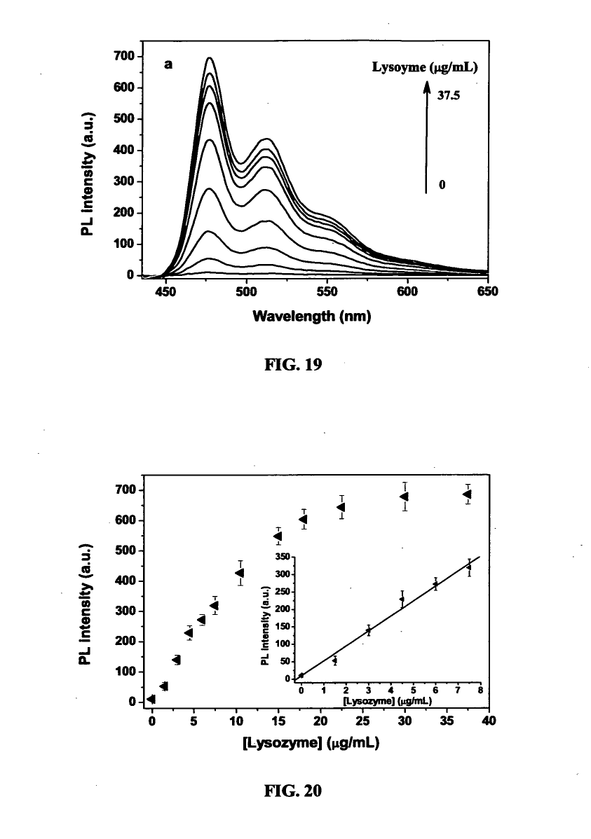

To demonstrate lysozyme quantification, different concentrations of lysozyme (ranging from 0 to 37.5 μg/mL) were incubated with 60 Apt-NP suspension for 30 minutes. The lysozyme/Apt-NPs were then stained with 1 μM PFVSO₃ for 5 minutes, then washed. The PL spectra of polymer-stained NPs are shown in FIG. 19. The PL intensities of the NPs progressively increase with increased lysozyme concentrations. This is due to increased positive charge on the Apt-NP surface in the presence of higher lysozyme concentrations, which enables increased numbers of negatively charged PFVSO₃ to self-assemble on the NPs. In addition, the fluorescence of the NP suspension upon treatment with lysozyme and PFVSO₃ can be monitored by the naked eye. The intensity of the blue-green fluorescence of PFVSO₃ gradually increases in the presence of increased concentrations of lysozyme, which allows clear naked-eye discrimination of lysozyme with a limit of detection (LOD) as low as 1.5 μg/mL (10 pmol).

The calibration curve for lysozyme detection is shown in FIG. 20. The PL intensity of the NP suspension increases linearly with lysozyme concentration and finally saturates at a lysozyme concentration of approximately 22.5 μg/mL. The LOD is estimated to be 0.36 μg/mL (2.4 μmol, based on 3σ from six independent measurements) using a standard fluorometer, which is more sensitive to aptamer-based electrochemical and fluorescent arrays and is similar to that obtained from a standard of ELISA. (See Vidal, M. L., et al., Agric. Food Chem. 2005, 53, 2379-2385, the entire teachings of which are incorporated herein by reference). However, the strategy of using Apt-NP as a platform for lysozyme detection reduces the bonding affinity (K_{d}) of the aptamer to its target. The apparent K_{d} in our assay is approximately 9 μg/mL (approximately 625 nM), which is estimated from the lysozyme concentration that induces half-maximum signal in FIG. 20. Similar to that of aptamer-immobilized gold assays, this K_{d} value is 20-fold larger compared to that measured in solution (31 nM). (See, for example, Cox, J. C.; Ellington, A. D., Bioorg. Med. Chem. 2001, 9, 2525-2531, the entire teachings are incorporated herein by reference). The large K_{d} on the NP surface is detrimental to assay sensitivity, could be the result of: (1) the steric hindrance induced by the folded aptamer upon binding to lysozyme which prevents the adjacent aptamers from folding into G-quartet structure; (2) the binding of lysozyme on the Apt-NP surface hampers subsequent aptamer/lysozyme binding due to electrostatic repulsion.

Although quite a few strategies have been reported for lysozyme detection, very few allow label-free and visible detection and quantification of lysozyme in real time.

### Example 16

### Synthesis of POSSFF and POSSFBT

### Synthesis of 2-(9,9-bis(6-bromohexyl)fluoren-2-yl)-4,4,5,5-tetramethyl-1,3,2-dioxaborolane (1)

2-Bromo-9,9-bis(6-bromohexyl)fluorene (4.54 g, 7.95 mmol), bis(pi nacolatodiboron) (3.02 g, 11.93 mmol), and potassium acetate (2.94 g, 29.82 mmol) were placed in a 100-mL round bottom flask. Anhydrous dioxane (80 mL) and [PdCl2(dppf)] (0.20 g, 0.24 mmol) were added to the flask and the reaction vessel was degassed. The mixture was stirred at 80° C. for 12 h under nitrogen. After the mixture had been cooled to room temperature, dioxane was removed by rotary evaporation. The residue was extracted with dichloromethane, and the organic phase was washed with water and brine, and dried over magnesium sulfate. The solvent was removed and the residue was purified by silica gel column chromatography (dichloromethane/hexane=1:2) to afford 2.

### Synthesis of 2,7-dibromo-9,9-bis(6-bromohexyl)fluorene (2)

2,7-Dibromofluorene (1.23 g, 5 mmol) was added to a mixture of aqueous potassium hydroxide (100 mL, 50 w %), tetrabutylammonium bromide (0.330 g, 1 mmol), and 1,2-bis(2-bromoethoxy)ethane (13.9 g, 50 mmol) at 75° C. After 15 min, the mixture was cooled to room temperature. After extraction with CH₂Cl₂, the combined organic layers were washed successively with water, aqueous HCl (1 M), water, and brine and then dried over Na₂SO₄. After removal of the solvent and the excess 1,2-bis(2-bromoethoxy)ethane, the residue was purified by silica gel column chromatography using hexane and dichloromethane (1:2) as the eluent, and recrystallized from ethanol and CH₂Cl₂(5:1) to afford M2 as white needle crystals (1.50 g, 48.0%).

### Synthesis of 2-(7-bromo-9,9-bis(6-bromohexyl)fluorenyl)-9,9-bis(6-bromohexyl)fluorene (3)

1 (2.84 g, 4.60 mmol), 2 (4.5 g, 6.9 mmol), Pd(PPh₃)₄ (53 mg, 0.046 mmol), potassium carbonate (4.43, 32.0 mmol) were placed in a 100 mL round bottom flask. A mixture of water (12 mL) and toluene (30 mL) was added to the flask and the reaction vessel was degassed. The mixture was vigorously stirred at 90° C. for 2 days. After it was cooled to room temperature, dichloromethane was added to the reaction mixture. The organic portion was separated and washed with brine before drying over anhydrous MgSO₄. The solvent was evaporated off, and the solid residues were purified by column chromatography on silica gel using dichloromethane/hexane (1:5) as eluent to afford 3.

### Synthesis of 2-(7-bromo-bis(6-N,N,N-trimethylammonium)hexyl)fluorenyl)-bis(6-N,N,N-trimethylammonium)hexyl)fluorene (4)

Condensed trimethylamine (˜5 mL) was added dropwise to a solution of 3 (1 g, 0.94 mmol) in THF (10 mL) at −78° C. The mixture was allowed to warm to room temperature. The precipitate was redissolved by the addition of water (10 mL). After the mixture was cooled to −78° C., additional trimethylamine (˜3 mL) was added. The mixture was stirred at room temperature for 24 h. After removal of the solvent, acetone was added to precipitate 4 (1.2 mg, 98%) as white powders.

### Synthesis of 4-(9,9-bis(6-bromohexyl)-9H-fluoren-2-yl)-7-bromobenzothiadiazole (7)

2-(9,9-bis(6-bromohexyl)-fluoren-2-yl)-4,4,5,5-tetramethyl-1,3,2-dioxaborolane (6) (2.84 g, 4.60 mmol), 4,7-dibromobenzothiadiazole (2.16 g, 7.36 mmol), Pd(PPh₃)₄ (53 mg, 0.046 mmol), potassium carbonate (4.43, 32.0 mmol) were placed in a 100 mL round bottom flask. A mixture of water (12 mL) and toluene (30 mL) added to the flask and the reaction vessel was degassed. The mixture was vigorously stirred at 90° C. for 2 days. After it was cooled to room temperature, dichloromethane was added to the reaction mixture. The organic portion was separated and washed with brine before drying over anhydrous MgSO₄. The solvent was evaporated off, and the solid residues were purified by column chromatography on silica gel using dichloromethane/hexane (1:5) as eluent to afford as grassy liquid. ¹H NMR (500 MHz, CD₃OD, δ ppm): 8.0-7.87 (m, 3H), 7.85 (d, 1H, J=7.84), 7.77 (d, 1H, J=7.26), 7.66 (d, 1H, J=7.57), 7.45-7.30 (m, 3H), 3.27 (t, 4H, J=6.84, 6.84), 2.14-1.97 (m, 4H), 1.74-1.62 (m, 4H), 1.32-1.18 (m, 4H), 1.17-1.04 (m, 4H), 0.83-0.66 (m, 4H). ¹³C NMR (125 MHz, CD₃OD, δ ppm): 154.00, 153.35, 152.83, 150.90, 141.76, 140.50, 135.37, 134.49, 132.31, 128.24, 128.05, 127.58, 127.08, 123.79, 122.91, 120.13, 119.89, 112.81, 55.16, 40.12, 33.92, 32.60, 29.04, 27.73, 23.61. MS (MALDI-TOF): m/z 707.37 [M]⁺.

### Synthesis of 4-(9,9-bis(6-N,N,N-trimethylammonium)hexyl)fluorenyl)-7-bromobenzothiadiazole (8)

Synthesis of Condensed trimethylamine (˜5 mL) was added dropwise to a solution of 2 (1 g, 0.94 mmol) in THF (10 mL) at −78° C. The mixture was allowed to warm to room temperature. The precipitate was redissolved by the addition of water (10 mL). After the mixture was cooled to −78° C., additional trimethylamine (˜3 mL) was added. The mixture was stirred at room temperature for 24 h. After removal of the solvent, acetone was added to precipitate 3 (1.4 mg, 99%) as yellow powders. ¹H NMR (500 MHz, CD₃OD, δ ppm): 8.38-8.26 (m, 2H), 8.26-8.19 (m, 1H), 8.19-8.12 (m, 1), 8.12-8.00 (m, 2H), 7.79-7.56 (m, 3H), 3.53-3.42 (m, 4H), 3.09 (3, 18H), 2.55-2.42 (m, 4H), 1.95-1.72 (m, 4H), 1.53-1.31 (m, 8H), 1.12-0.78 (m, 4H). (¹³C NMR (125 MHz, CD₃OD, δ ppm): 155.28, 154.50, 152.26, 152.055, 143.31, 142.18, 136.97, 135.38, 134.03, 129.73, 128.93, 128.46, 125.18, 124.33, 121.35, 121.05, 113.78, 67.81, 55.58, 53.68, 41.19, 30.35, 26.98, 24.92, 23.75.

Synthesis of POSSFF.

Octavinyl POSS (5) (11.4 mg, 0.018 mmol), 4 (187 mg, 0.144 mmol), Pd(OAc)₂ (3.2 mg, 14.4 μmol), and P(o-tolyl)₃ (24 mg, 78.4 μmol) were placed in a 25 mL round bottom flask. A mixture of DMF (1 mL), and triethylamine (0.5 mL) was added to the flask and the reaction vessel was degassed. The mixture was vigorously stirred at 100° C. for 36 h. It was then filtered and the filtrate was poured into acetone. The precipitate was collected and washed with acetone, and was redissolved in water. The solution was filtered through a 0.22 μm syringe driven filter to give limpid solution. Finally, the product was purified by dialysis against Milli-Q water using a 3.5 kDa molecular weight cutoff dialysis membrane for 5 days. After freeze-drying, POSSFF (74 mg, 45%) was obtained as light yellow powders.

Synthesis of POSSFBT.

Octavinyl POSS (5) (11.4 mg, 0.018 mmol), 8 (119 mg, 0.144 mmol), Pd(OAc)₂ (3.2 mg, 14.4 μmol), and P(o-tolyl)₃ (24 mg, 78.4 μmol) were placed in a 25 mL round bottom flask. A mixture of DMF (1 mL), and triethylamine (0.5 mL) was added to the flask and the reaction vessel was degassed. The mixture was vigorously stirred at 110° C. for 36 h. It was then filtered and the filtrate was poured into acetone. The precipitate was collected and washed with acetone, and was redissolved in water. The solution was filtered through a 0.22 μm syringe driven filter to give limpid solution. Finally, the product was purified by dialysis against Milli-Q water using a 3.5 kDa molecular weight cutoff dialysis membrane for 5 days. After freeze-drying, POSSBT (96 mg, 73%) was obtained as yellow fibers. ¹H NMR (500 MHz, CD₃OD, δ ppm): 8.47 (s, 1H), 8.43 (d, 2H), 8.31 (d, 1H), 8.25 (d, 2H), 7.74-7.76 (m, 2H), 7.83-7.74 (m, 1H), 7.74-7.63 (m, 2H), 3.54-3.38 (m, 4H), 3.09 (s, 18H), 2.57-2.39 (m, 4H), 1.95-1.80 (m, 4H), 1.54-1.40 (m, 8H), 1.13-0.95 (m, 4H).

This unimolecular nanoparticle has a good water-solubility (˜23 mg/mL at 24° C.), as a result of its high charge density on its nanoglobular surface. The morphology and size of POSSFBT were studied by high-resolution transmission electron microscopy (HR-TEM). Spherical nanoparticles with an average diameter of 3.3±0.5 nm were observed, which coincides well with the single-molecular size of POSSFBT.

POSS compounds containing catonic, anionic or neutral R groups on either Ar or Ar′ can be synthesized by the similar method as that used to synthesize POSSFF and POSSFBT.

1. A compound represented by the following structural formula: 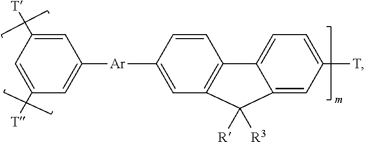
or a salt thereof; wherein:
R′ and R³ are each independently hydrogen or a charged side group;
m is an integer between 2 and 50, inclusive;
Ar is an optionally substituted monocyclic or polycyclic aromatic ring system or an optionally substituted monocyclic or polycyclic heteroaromatic ring system; and
T, T′ and T″ are each independently a terminating group, -L or -L′-B, wherein L and L′ are each independently a linking group and wherein B, for each occurrence, is a biomolecule.

2. The compound of claim 1, or a salt thereof, wherein R′ and R³ are each a cationic alkyl group or a cationic oligo or poly(ethylene oxide) group.

3. The compound of claim 1, or a salt thereof, wherein R′ and R³ are each a charged side group selected from the group consisting of —(CH₂)ₙN(R²)₃X, —(OCH₂CH₂)ₙN(R²)₃X and —(CH₂CH₂O)_{q}CH₂CH₂N(R²)₃X, wherein R² is (C1-C6)alkyl, n is an integer between 2 and 13, inclusive, q is an integer between 1 and 12, inclusive, and X is an anionic counterion.

4. The compound of claim 1, or a salt thereof, wherein Ar is fluorene, benzene, biphenyl, thiophene, benzothiadiazole, pyridine, bipyridinium, triphenylamine, anthracene, carbazole or 4,7-di(thien-5′-yl)-2,1,3-benzothiadiazole.

5. (canceled)

6. The compound of claim 1, or a salt thereof, wherein T, T′ and T″ are each -L′-B, wherein B, for each occurrence, is independently a biomolecule.

7. The compound of claim 1, or a salt thereof, wherein T, T′ and T″ are each -L′-B, wherein each B is a biomolecule.

8. The compound of claim 1, or a salt thereof, wherein B, for each occurrence, is independently a ligand or a reporter group.

9. (canceled)

10. The compound of claim 1, wherein the compound is represented by the following structural formula:

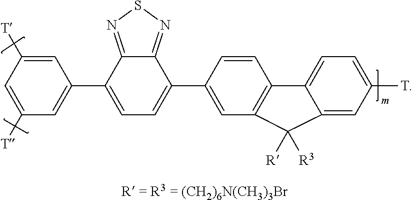

11. The compound of claim 1, or a salt thereof, wherein L is represented by one of the following structural formulas: 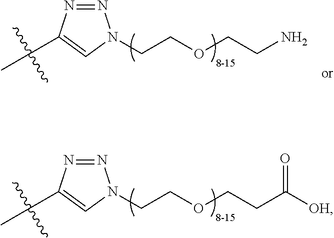
or a salt thereof.

12. The compound of claim 1, or a salt thereof, wherein L′ is represented by one of the following structural formulas:

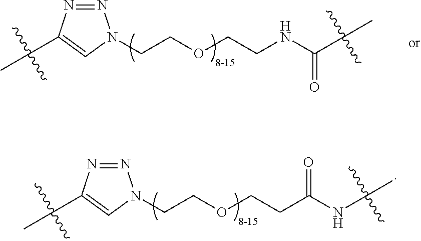

13. A compound represented by the following structural formula: 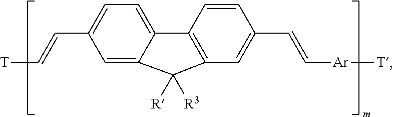
or a salt thereof; wherein:
R′ and R³ are each independently hydrogen or a charged side group, wherein the charged side group is optionally functionalized with -L or -L′-B, wherein L and L′ are each independently a linking group and wherein B, for each occurrence, is independently a biomolecule;
m is an integer between 2 and 50, inclusive;
Ar is an optionally substituted monocyclic or polycyclic aromatic ring system or an optionally substituted monocyclic or polycyclic heteroaromatic ring system; and
T and T′ are each independently a terminating group.

14. The compound of claim 13, or a salt thereof, wherein R′ and R³ are each a charged side group functionalized with -L or -L′-B, wherein L and L′ are each a linking group and wherein each B is a biomolecule.

15. The compound of claim 14, or a salt thereof, wherein each B is a ligand or a reporter group.

16. (canceled)

17. The compound of claim 13, or a salt thereof, wherein Ar is fluorene, benzene, biphenyl, thiophene, benzothiadiazole, pyridine, bipyridinium, triphenylamine, anthracene, carbazole or 4,7-di(thien-5′-yl)-2,1,3-benzothiadiazole.

18. The compound of claim 13, wherein the compound is represented by the following structural formula:

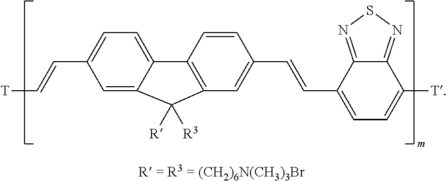

19. The compound of claim 13, or a salt thereof, wherein L is represented by one of the following structural formulas:

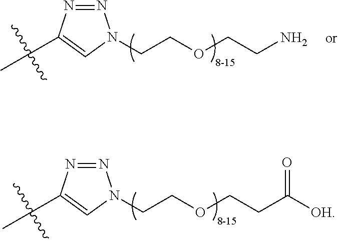

20. The compound of claim 13, or a salt thereof, wherein L′ is represented by one of the following structural formulas:

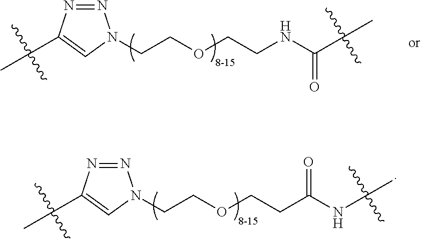

21. A compound of the following structural formula: 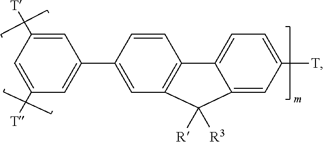
or a salt thereof; wherein:
R′ and R³ are each independently hydrogen or a charged side group;
m is an integer between 2 and 50, inclusive; and
T, T′ and T″ are each independently a terminating group, -L or -L′-B, wherein L and L′ are each independently a linking group and wherein B, for each occurrence, is independently a biomolecule.

22. The compound of claim 21, or a salt thereof, wherein the R′ and R³ are each a charged side group.

23. The compound of claim 22, or a salt thereof, wherein the charged side group is a cationic alkyl group, a cationic oligo or poly(ethylene oxide) group, an anionic alkyl group or an anionic oligo or poly(ethylene oxide) group.

24. The compound of claim 23, or a salt thereof, wherein the charged side group is —(CH₂)₂COOH.

25. A method of detecting a target in a sample, comprising:
a) exposing a sample to a compound of one of the following structural formulas: 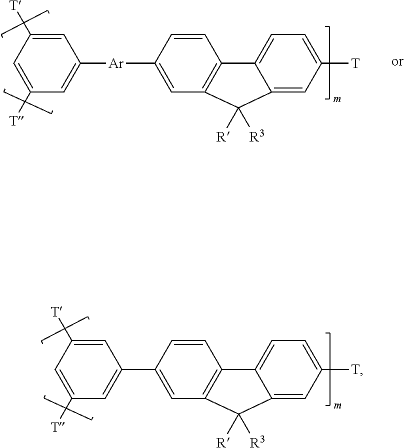
or a salt thereof; wherein:
R′ and R³ are each independently hydrogen or a charged side group;
m is an integer between 2 and 50, inclusive;
Ar is an optionally substituted monocyclic or polycyclic aromatic ring system or an optionally substituted monocyclic or polycyclic heteroaromatic ring system; and
T, T′ and T″ are each independently a terminating group, -L or -L′-B, wherein L and L′ are each independently a linking group and B, for each occurrence, is independently a biomolecule; or 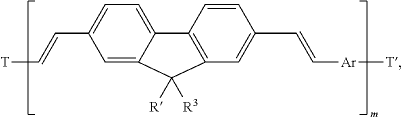
or a salt thereof; wherein:
R′ and R³ are each independently hydrogen or a charged side group, wherein the charged side group is optionally functionalized with -L or -L′-B, wherein L and L′ are each independently a linking group and B, for each occurrence, is independently a biomolecule;
m is an integer between 2 and 50, inclusive;
Ar is an optionally substituted monocyclic or polycyclic aromatic ring system or an optionally substituted monocyclic or polycyclic heteroaromatic ring system; and
T and T′ are each independently a terminating group;
b) allowing the compound to bind to a target; and
c) detecting a signal produced by the compound, thereby detecting the target.

26.-27. (canceled)

28. The compound of claim 13, represented by Structural Formula (III): 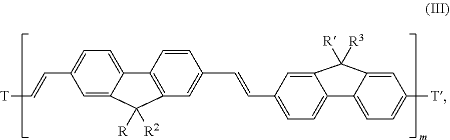
or a salt thereof, wherein:
R and R² are each independently —(OCH₂CH₂)ₚOCH₃ or —(CH₂CH₂O)ₚCH₃, wherein p is an integer between 1 and 100, inclusive;
R′ and R³ are each independently hydrogen or a charged side group;
m is an integer between 2 and 50, inclusive; and
T and T′ are each independently a terminating group.

29. The compound of claim 28, represented by Structural Formula (IIIa): 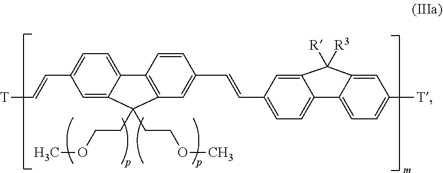
or a salt thereof.

30. A method of detecting a target in a sample, comprising:
functionalizing a solid support with a ligand;
incubating the ligand-functionalized solid support with a sample;
incubating the sample with a charged conjugated polyelectrolyte (CPE) or conjugated oligoelectrolyte (COE); and
detecting the fluorescence of the solid support, thereby detecting the target.

31. The method of claim 30, wherein the CPE or COE is represented by one of the following structural formulas, or a salt thereof: 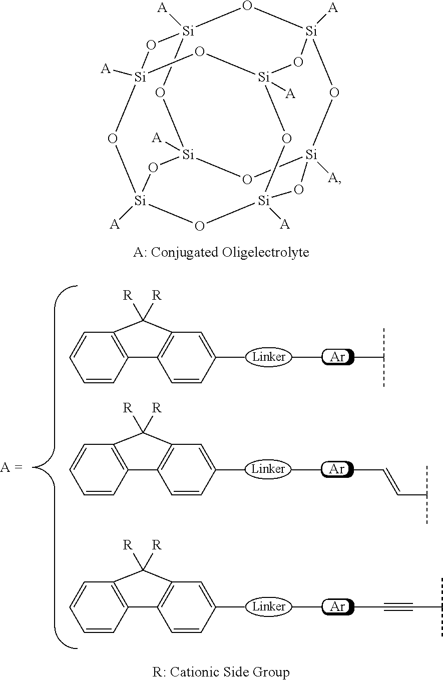
wherein:
Ar is an optionally substituted aromatic group;
Linker is a single bond, double bond, triple bond or —CR¹₂—; wherein each R¹ is independently hydrogen, halogen, hydroxy, amino, C₁-C₆alkyl, C₁-C₆alkenyl, C₁-C₆alkynyl, or C₁-C₆alkoxy; wherein the alkyl, alkenyl, alkynyl or alkoxy may be optionally substituted with halogen, hydroxy, C₁-C₄alkoxy or amino; and
each R is independently hydrogen, a cationic alkyl side group or a cationic oligo or poly(ethylene oxide) group; 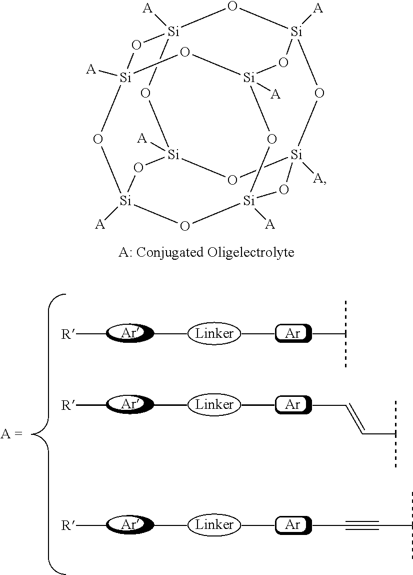
wherein:
each 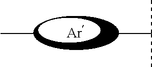
is independently selected from: 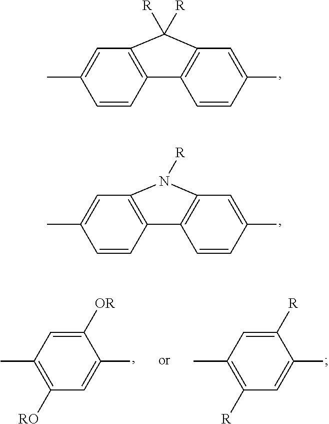
each Ar is independently an optionally substituted aromatic group;
each R is independently a cationic, anionic, or neutral alkyl group or a cationic, anionic, or neutral oligo or poly(ethylene oxide) group;
each Linker is a single bond, double bond, triple bond, —CH₂— or —CH₂CH₂—; and
each R′ is independently a terminating group; 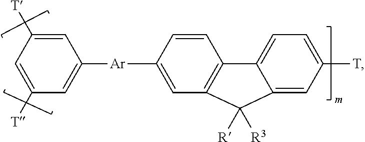
wherein:
R′ and R³ are each independently hydrogen or a charged side group;
m is an integer between 2 and 50, inclusive;
Ar is an optionally substituted monocyclic or polycyclic aromatic ring system or an optionally substituted monocyclic or polycyclic heteroaromatic ring system; and
T, T′ and T″ are each independently a terminating group; or 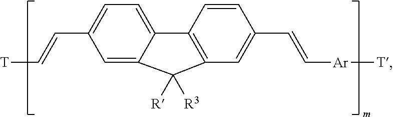
wherein:
R′ and R³ are each independently hydrogen or a charged side group;
m is an integer between 2 and 50, inclusive;
Ar is an optionally substituted monocyclic or polycyclic aromatic ring system or an optionally substituted monocyclic or polycyclic heteroaromatic ring system; and
T and T′ are each independently a terminating group.

32. (canceled)
